(19) United States
(12) Patent Application Publication
(10) Pub. No.: US 2024/0105339 A1
(43) Pub. Date: Mar. 28, 2024

(54) PASSIVE BEHAVIORAL HEALTH VITAL SIGNS

(71) Applicant: HEALTH RHYTHMS, INC., Long Island City, NY (US)

(72) Inventors: MARK MATTHEWS, DUBLIN (IE); GABRIEL ARANOVICH, NEW YORK, NY (US); GREGORY POSEY, VERPLANCK, NY (US); MCKAY LARSON, WINDSOR, VT (US); SAMUEL A. BURGESS, NEW YORK, NY (US); JEREMY LEACH, Long Island City, NY (US)

(21) Appl. No.: 18/083,014

(22) Filed: Dec. 16, 2022

Related U.S. Application Data
(60) Provisional application No. 63/409,543, filed on Sep. 23, 2022.

(51) Int. Cl. G16H 50/30; G16H 10/20; G16H 10/60; G16H 50/20

(52) U.S. Cl. CPC G16H 50/30; G16H 10/20; G16H 10/60; G16H 50/20

(57) ABSTRACT

In an embodiment, a computer-implemented method comprises receiving at a server computer via a data communication network, a plurality of raw sensor data and a plurality of self-report score values from a plurality of mobile computing devices; using the server computer, for a particular mobile computing device from among the plurality of mobile computing devices; calculating a plurality of inference data based upon transformations of the raw sensor data and the plurality of self-report score values; the server computer accessing via the network a patient electronic health record that is associated with a user of the particular mobile computing device; inputting the raw sensor data and the electronic health record to a plurality of trained machine learning models; operating the plurality of trained machine learning models in an inference phase to generate outputs specifying probabilities that a user of the particular mobile computing device would respond above a particular threshold for each item of the patient health questionnaire; based on the probabilities, the server computer calculating a risk value of the user, updating a graphical user interface of a healthcare provider computer to display the risk value, and based on the risk value, and transmitting one or more of an on-call staff alert or a welfare check request.

### BENEFIT CLAIM

This application claims the benefit under 35 U.S.C. § 119(e) of provisional application 63/409,543, filed Sep. 23, 2022, the entire contents of which are hereby incorporated by reference for all purposes as if fully set forth herein.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright or rights whatsoever. © 2022 Health Rhythms, Inc.

### TECHNICAL FIELD

One technical field of the present disclosure is relational database systems, including generating synthetic data to supplement sparse datasets and automated inferential transformations of database records.

### BACKGROUND

The approaches described in this section are approaches that could be pursued, but not necessarily approaches that have been previously conceived or pursued. Therefore, unless otherwise indicated, it should not be assumed that any of the approaches described in this section qualify as prior art merely by virtue of their inclusion in this section.

Computer-assisted tools have become available for the automated measurement of mental health conditions. However, at present, existing systems rely on the use of periodic questionnaires, visits to websites, phone calls or video conferences, other telehealth techniques, or clinical visits, for collecting data for assessment. These techniques necessarily collect data at discrete points, with long intervals between collections. But behavioral health is known to vary on a continuous basis. Collecting information to appraise an individual's behavioral health continuously is crucial to directing care and understanding factors relevant to a condition and risk factors relating to new conditions. Furthermore, existing systems are cumbersome and often collect data consisting more of noise than signal. Consequently, there is an acute need in the field of mental health diagnosis and treatment for improved machine-implemented methods of continuously collecting and evaluating measurements of individualized data pertaining to behavioral health.

### SUMMARY

The appended claims may serve as a summary of the invention.

### BRIEF DESCRIPTION OF THE DRAWINGS

In the drawings:

FIG. 1A and FIG. 1B illustrate a distributed computer system showing the context of use and principal functional elements with which one embodiment could be implemented.

FIG. 2 illustrates example sources of sensor data and inferential transformation of the sensor data.

FIG. 3 illustrates an example of the transformation of inference data into an example digital questionnaire directed to a subject.

FIG. 4 illustrates an example of the transformation of inference data into digital suggestions that can be created and stored for transmission to a subject.

FIG. 5 illustrates an example data flow in which specified data sources are transformed into a stratum of risk values to update a clinical dashboard.

FIG. 6 illustrates an example of a workflow that is programmed to collect data, stratify the attention of a care team concerning a plurality of subjects, and initiate responses using other systems.

FIG. 7 illustrates a computer system with which one embodiment could be implemented.

FIG. 8A and FIG. 8B illustrate portions of an example of a care team interface, in one embodiment.

FIG. 9A illustrates a portion of an example of a care team interface in which certain graphical panels are collapsed.

FIG. 9B illustrates a portion of an example of a care team interface in which certain graphical panels are expanded.

### DETAILED DESCRIPTION

In the following description, for the purposes of explanation, numerous specific details are set forth to provide a thorough understanding of the present invention. It will be apparent, however, that the present invention may be practiced without these specific details. In other instances, well-known structures and devices are shown in block diagram form to avoid unnecessarily obscuring the present invention.

The text of this disclosure, in combination with the drawing figures, is intended to state in prose the algorithms that are necessary to program the computer to implement the claimed inventions, at the same level of detail that is used by people of skill in the arts to which this disclosure pertains to communicate with one another concerning functions to be programmed, inputs, transformations, outputs and other aspects of programming. That is, the level of detail set forth in this disclosure is the same level of detail that persons of skill in the art normally use to communicate with one another to express algorithms to be programmed or the structure and function of programs to implement the inventions claimed herein.

### 1. General Overview

In one embodiment, the disclosure provides a client-server distributed computing system that is programmed for collecting patient self-reported information and passive data that is collected from a plurality of sensors, which can be embedded in smartphones and other devices and processing the data to yield summaries that represent an individualized functional profile in terms of behavioral health vital signs. The behavioral vital signs can be related to a person's sleep, social activity, physical activity, overall routine, and other areas of behavior with the intention of assessing individual behavior and mental health. In an embodiment, data summaries can be presented in any of a plurality of different interfaces. For example, one interface may comprise a clinician dashboard, a second interface may be tailored to a clinician within an electronic health record (EHR), and a third interface may be optimized for consumption by a patient or care provider. In an embodiment, the data summaries can be continuously updated in real-time in response to receiving additional periodic self-reported data from the subject or additional sensor or health record data on the subject.

The disclosed techniques allow clinicians and other systems or algorithms to receive aggregated and summarized data that interprets or represents a person's behavioral health on an ongoing basis including the current overall state, the status of individual areas of behavioral health, trends over various time frames, and clinical significance of individual areas and overall status. In areas of an individual's behavior that might need clinical attention, act as a guide to deciding which treatment might be most appropriate for a particular patient (e.g., if they are not sleeping then it might make sense to prescribe a digital sleep program) AND whether prescribed treatments are having the desired impact on the target behavior. In some embodiments, the data summaries can be programmatically transferred to other systems for further downstream processing.

Various embodiments encompass the subject matter of one or more of the following numbered clauses:
1. A computer-implemented method comprising: receiving at a server computer via a data communication network, a plurality of raw sensor data and a plurality of self-report score values from a plurality of mobile computing devices; using the server computer, for a particular mobile computing device from among the plurality of mobile computing devices; calculating a plurality of inference data based upon transformations of the raw sensor data and the plurality of self-report score values; the server computer accessing via the network a patient electronic health record that is associated with a user of the particular mobile computing device; inputting the raw sensor data and the electronic health record to a plurality of trained machine learning models; operating the plurality of trained machine learning models in an inference phase to generate outputs specifying probabilities that a user of the particular mobile computing device would respond above a particular threshold for each item of the patient health questionnaire; based on the probabilities, the server computer calculating a risk value of the user, updating a graphical user interface of a healthcare provider computer to display the risk value, and based on the risk value, and transmitting one or more of an on-call staff alert or a welfare check request.
2. The computer-implemented method of clause 1, the inference data comprising one or more of a Behavioral Health Score, Sleep Score, Social Score, Routine Score, and Physical Activity Score.
3. The computer-implemented method of clause 1, the plurality of trained machine learning models each comprising an ensemble of decision trees that have been trained using training data to track a particular item on a patient health questionnaire for depression.
4. The computer-implemented method of clause 3, the training data comprising a plurality of records each comprising attribute values for patient demographics and one or more inferences or behavioral summaries that have been derived from the raw sensor data, and one or more sets of responses to items of the patient health questionnaire.
5. The computer-implemented method of clause 4, the training data comprising, for a particular patient that is associated with the particular mobile computing device, a plurality of sets of responses to the patient health questionnaire corresponding to a plurality of different days.
6. The computer-implemented method of clause 4, the training data comprising, for a particular patient that is associated with the particular mobile computing device, a plurality of sets of responses to the PHQ-8 patient health questionnaire for depression and corresponding to a plurality of different days.
7. The computer-implemented method of clause 3, further comprising, based on the inference data, selecting a digitally stored different questionnaire, transmitting the different questionnaire to the particular mobile computing device, receiving a plurality of responses corresponding to plural items in the different questionnaire, and updating the training data based on the responses.
8. The computer-implemented method of clause 1, each model among the plurality of trained machine learning models comprising a classification model that is associated with a particular item of the patient health questionnaire, each classification model being configured to output a probability that, if given the patient health questionnaire, a response of a patient would exceed a particular threshold value for the particular item.
9. The computer-implemented method of clause 8, the plurality of trained machine learning models comprising pairs of models, each of the pairs being associated with a common category of gender and activity, each of the models being trained on a particular item of the PHQ-8 patient health questionnaire for depression.
10. The computer-implemented method of clause 9, the particular item comprising any of item “1”, “2”, “3”, or “4” of the PHQ-8 patient health questionnaire for depression.
11. The computer-implemented method of clause 1, further comprising, based on the inference data, selecting and transmitting to the particular mobile computing device a plurality of suggestion messages each comprising a text suggestion for presentation on the particular mobile computing device.
12. The computer-implemented method of clause 1, the plurality of raw sensor data comprising location data, pedometer data, activity data, and device data, the device data specifying any of device unlock events, device lock events, device lock screen display events, application launch events, application dismiss events.
13. The computer-implemented method of clause 1, the activity data specifying physical interactions of a user with the particular mobile computing device including one or more gestures, taps, operation of hardware switches or controls.
14. One or more non-transitory computer-readable data storage media storing one or more sequences of instructions which when executed using one or more processors cause the one or more processors to execute according to the subject matter of any of clause 1 to clause 13, inclusive.
15. A computer system comprising one or more processors, a memory coupled to the one or more processors, and one or more non-transitory computer-readable data storage media coupled to the one or more processors and storing one or more sequences of instructions which when executed using one or more processors cause the one or more processors to execute according to the subject matter of any of clause 1 to clause 13, inclusive.

### 2. Structural & Functional Overview

2.1 Distributed Computer System Example

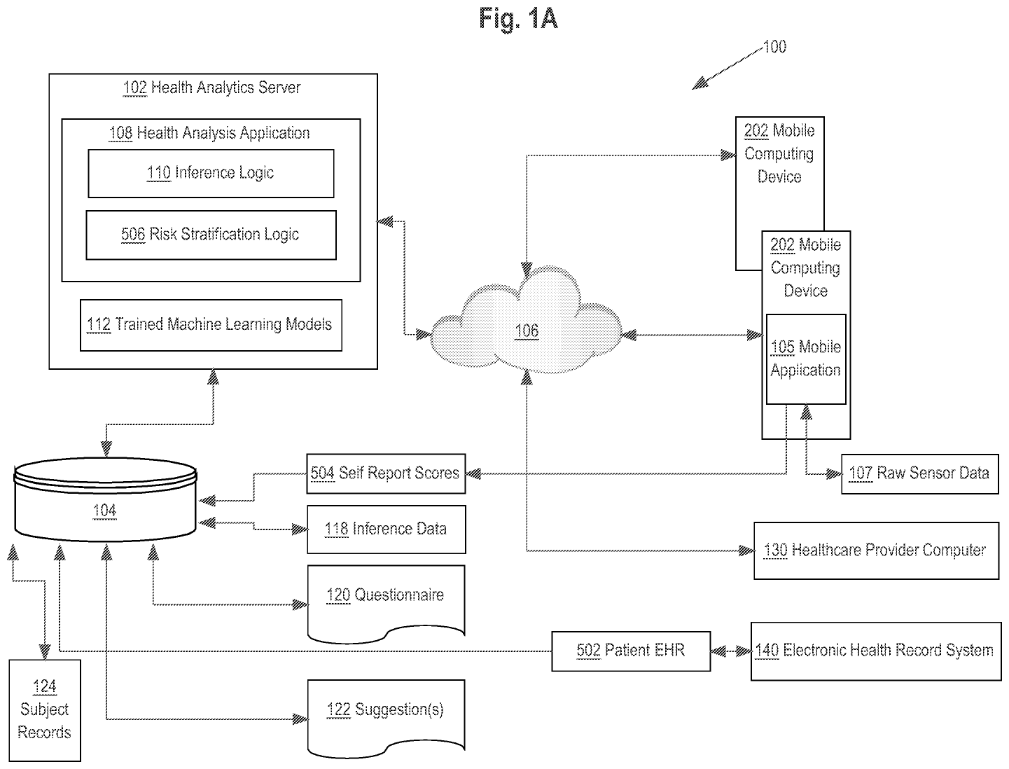

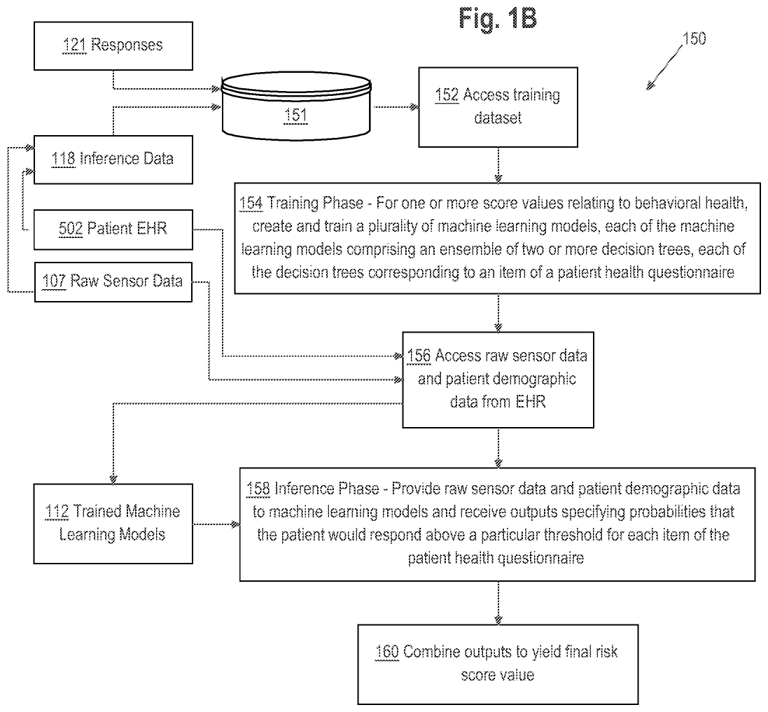

FIG. 1A illustrates a distributed computer system showing the context of use and principal functional elements with which one embodiment could be implemented. In an embodiment, a computer system 100 comprises components that are implemented at least partially by hardware at one or more computing devices, such as one or more hardware processors executing stored program instructions stored in one or more memories for performing the functions that are described herein. In other words, all functions described herein are intended to indicate operations that are performed using programming in a special-purpose computer or general-purpose computer, in various embodiments. FIG. 1A illustrates only one of many possible arrangements of components configured to execute the programming described herein. Other arrangements may include fewer or different components, and the division of work between the components may vary depending on the arrangement.

FIG. 1A, and the other drawing figures and all of the description and claims in this disclosure, are intended to present, disclose and claim a technical system and technical methods in which specially programmed computers, using a special-purpose distributed computer system design, execute functions that have not been available before to provide a practical application of computing technology to the problem of automatically collecting and analyzing social determinants of health, SDH, values and reporting results for use in supporting interventions or care. In this manner, the disclosure presents a technical solution to a technical problem, and any interpretation of the disclosure or claims to cover any judicial exception to patent eligibility, such as an abstract idea, mental process, method of organizing human activity or mathematical algorithm, has no support in this disclosure and is erroneous.

In one embodiment, a health analytics server 102 is communicatively coupled via network 106 to a plurality of different mobile computing devices 202, each hosting a mobile application 105 that is compatible with functional elements of the health analytics server. For purposes of illustrating a clear example, elements 202 are identified and described as mobile computing devices, but stationary computing devices also can be used, including but not limited to voice-activated digital assistants, desktop computers, connected televisions, or other fixed systems. In various embodiments, the health analytics server 102 can be deployed as a server computer, multiprocessor computer, or server cluster in a private data center, or as one or more virtual machine instances in any of a public or private data center. For example, one or more computing instances and storage instances of public commercial cloud computing centers like GOOGLE CLOUD, MICROSOFT AZURE, AMAZON WEB SERVICES can be used. The health analytics server 102 is communicatively coupled to a database 104 that is configured or programmed to digitally store inference data 118, one or more questionnaires 120 and responses to questionnaires, one or more suggestions 122, self-report scores 504, and subject records 124. The database 104 can be configured or organized as a relational database, object store, no-SQL database, or flat file system.

Each of the mobile computing devices 202 can be a laptop computer, desktop computer, tablet computer, smartphone, watch, or ring. Mobile computing device 202 host an operating system and include a plurality of hardware sensors, as further described in other sections. For purposes of illustrating a clear example, FIG. 1A shows two mobile computing devices, but in other embodiments, hundreds to millions of devices could be used depending upon the processing power of the health analytics server 102.

Network 106 broadly represents any combination of local area networks, wide area networks, campus networks, and/or internetworks using any of wired or wireless, satellite or terrestrial network links, and can include the public internet.

In an embodiment, the mobile device application or app 105 is programmed to passively collect and store raw sensor data 107 comprising digital signals relating to, for an individual: sleep; location; physical activity; device usage; metadata; diagnostic data; raw data summary; phone authorizations. Signals relating to the foregoing attributes or categories are digitally stored on-device in app memory. The mobile device app can be programmed to analyze the signal data and to detect vital clinical signals such as increased isolation, reduced mobility, and disrupted sleep, and to store the raw sensor data 107 on-device in app memory or app storage organized as a database, set of tables, or set of files. Or, the signal data can be collected, de-identified, encrypted, or otherwise secured, and transmitted via network 106 to the health analytics server 102. In either case, the collection, storage, and analysis of signal data is conducted on a de-identified basis to ensure that signal data, if compromised, cannot identify an individual user of the mobile computing device 202.

The health analytics server 102 can host or execute a health analysis application 108 and a plurality of trained machine learning models 112. The health analysis application 108 can be organized as one or more programs to execute the detection of clinical signals and stored in the database 104. The machine learning models 112 can be implemented as trained classifiers as detailed in other sections herein.

In one embodiment, the mobile device app 105 and/or health analysis application 108 is programmed to collect the foregoing data signals continuously and to provide alerts when significant deviations occur from personalized baselines, as well as trend indicators. In some embodiments, mobile app 105 can generate self-report scores 504 that contribute to the database 104 for use in data transformation or analytical operations. The mobile app 105 can be programmed to generate and display question sets or questionnaires to the mobile computing device 202, receive input from the user, and generate the self-report scores 504 for reporting over the network 106 to the health analytics server 102.

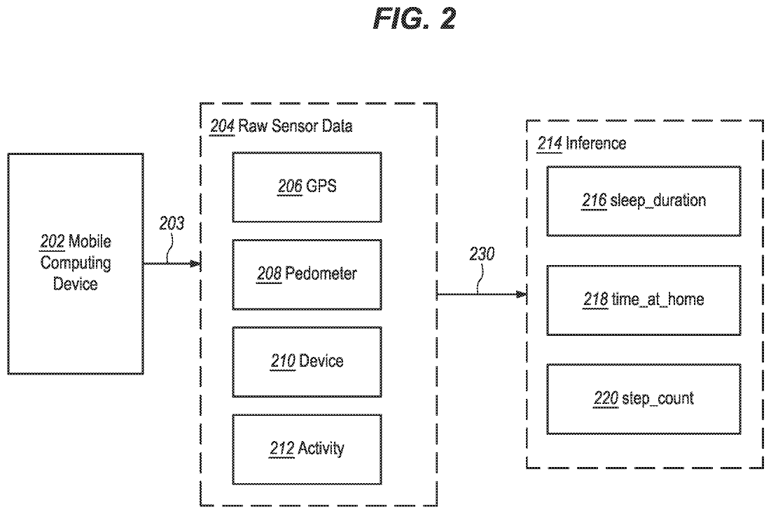

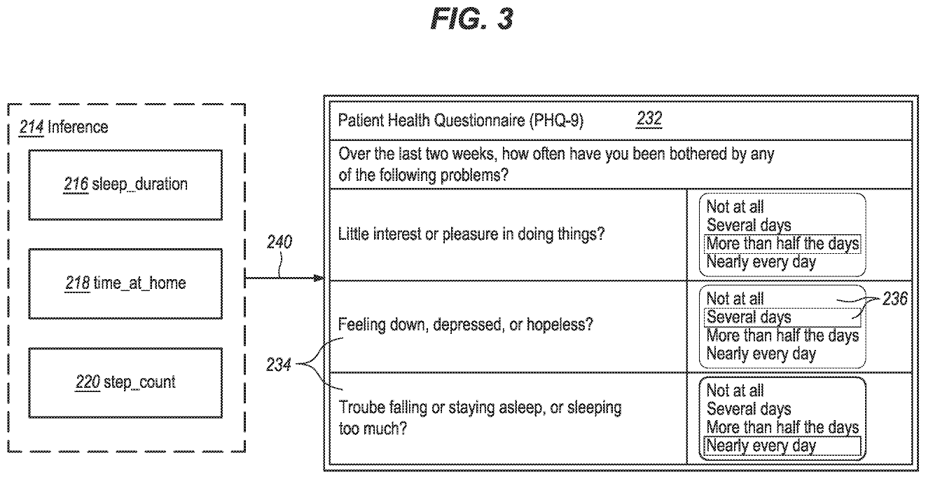

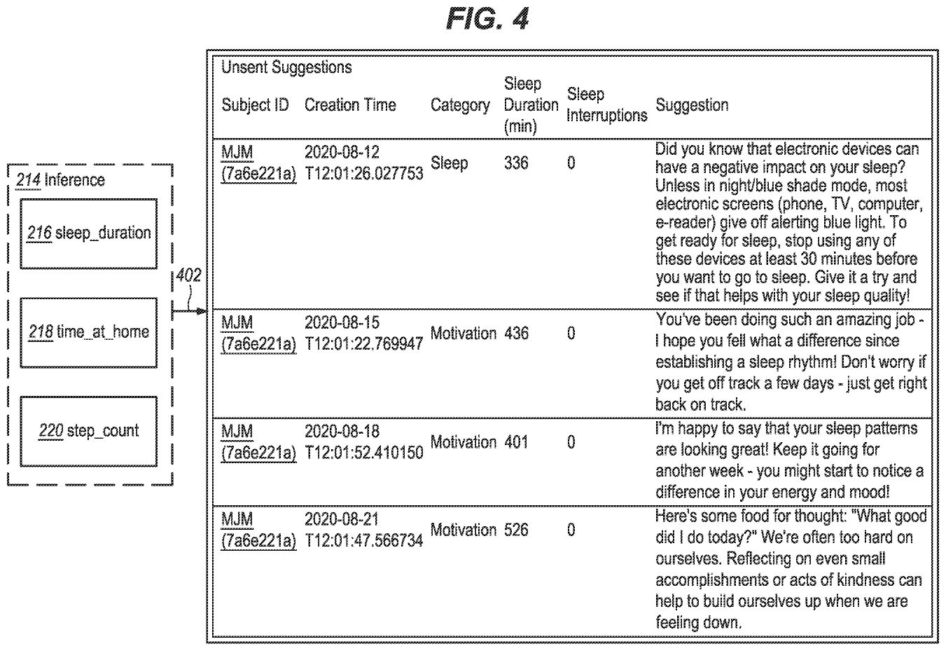

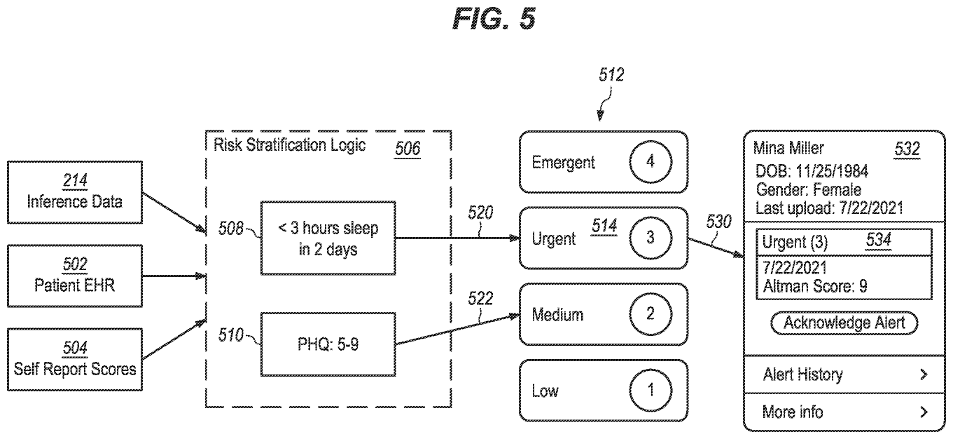

In an embodiment, health analysis application 108 comprises inference logic 110 and risk stratification logic 506, each of which can be implemented as a set of stored program instructions that implement the functions that are further described herein in other sections relating to FIG. 2, FIG. 3, FIG. 4, FIG. 5. In various embodiments, the inference logic 110 can be programmed to receive or read raw sensor data 107, execute one or more algorithms or methods to transform the raw sensor data, and produce inference data 118 as output for storage in the database 104. In an embodiment, the inference logic 110 is programmed to read the inference data 118 and transform the inference data into one or more questionnaires 120, and/or one or more suggestions 122.

In an embodiment, the risk stratification logic 506 is programmed to read inference data 118 and execute one or more algorithms or methods to transform the inference data into a score value selected from among a plurality of score values in a hierarchy or strata of risk values. A risk value derived or inferred for a particular subject or user of a mobile computing device 202 can be created and stored as an attribute of a subject record, among a plurality of subject records 124 stored in the database 104, corresponding to the user or subject.

A healthcare provider computer 130 can be communicatively coupled via network 106 to a health analytics server. The healthcare provider computer 130 can be used by or associated with a care team member who provides care to one or more users of the mobile computing devices 202. In an embodiment, health analytics server 202 is programmed to generate reports and views of the subject records 124 that include questionnaires 120, suggestions 122, inference data 118, score values, and other information or data that is transformed from or derived from the raw sensor data 107.

2.2 Functional Overview

FIG. 2 illustrates example sources of sensor data and inferential transformation of the sensor data. FIG. 2 and each other data flow diagram or process flow diagram herein is intended as an illustration at the functional level at which skilled persons, in the art to which this disclosure pertains, communicate with one another to describe and implement algorithms using programming. The flow diagrams are not intended to illustrate every instruction, method object or sub-step that would be needed to program every aspect of a working program but are provided at the same functional level of illustration that is normally used at the high level of skill in this art to communicate the basis of developing working programs.

In an embodiment, each mobile computing device 202 collects and stores, as arrow 203 indicates, raw sensor data 204 comprising location data 206, pedometer data 208, device data 210, and activity data 212. For purposes of illustrating a clear example, FIG. 2 shows four (4) sources of raw data, but other embodiments can include more or different sensors or sources. For example, various embodiments can use Bluetooth radio to connect to external devices and receive sensor data from those devices, barometers, and battery depletion sensors. Raw sensor data 204 can be stored using in-app memory of mobile application 105 in a mobile computing device 202. The location data 206 can comprise a plurality of geo-location data points, such as latitude-longitude pairs obtained using a GPS transceiver and/or location services of the mobile computing device. Thus, in various embodiments, location data 206 can comprise data values obtained directly from a GPS transceiver or other hardware source, or data indirectly representative of a geophysical position that system services generate. Location data 206 can be timestamped to associate a date-time value with each latitude-longitude (lat-long) pair or other values representing the location.

The pedometer data 208 can comprise a time series of magnitude values received from an accelerometer in the mobile computing device 202, in raw form or after processing using system services to identify data that appears to represent human step movement. The device data 210 can consist of or represent events that the mobile computing device 202 processes, including but not limited to device unlock events, device lock events, device lock screen display events, application launch events, application dismiss events, and so forth. The activity data 212 can consist of or represent physical interactions of a user with a mobile computing device, such as gestures, taps, operation of hardware switches or controls, and so forth.

In one embodiment, the mobile device app and/or server-side application program(s) are programmed to passively collect mobile device data and conduct on-device analysis of signals to yield one or more passive behavioral health summaries. In an embodiment, arrow 230 represents executing one or more inference algorithms that receive raw sensor data 204 as input, execute algorithmic or mathematical transformations of the data, and output inference data 214, which consists of a plurality of inferred values that interpret or add meaning to the raw sensor data. For example, various combinations of location data 206, pedometer data 208, device data 210, and activity data 212 can be transformed into a sleep_duration value 216 that represents a period of sleep of the user, a time_at_home value 218 that represents a period during which the user has been determined to be home. For purposes of illustrating a clear example, FIG. 2 shows three (3) inference values 216, 218, 220, but other embodiments can be programmed to generate and store different inferences or more inferences based on more or different raw sensor data 204 and various programmed algorithms or transformations.

TABLE 1 illustrates example inferences that can be programmed in various embodiments to provide high-level summaries of a person's behavioral health. In one embodiment, each of the summaries of TABLE 1 can be programmed to be calculated when about 10 days to 60 days of data has been collected; in one specific experimental embodiment, data collection for about 14 days has been found to suffice. Furthermore, in some embodiments, the mobile application 105 and/or inference logic 110 can interoperate with machine learning models that have been trained with historic data such that the trained models are available for use from the point of installation of the mobile application; such an embodiment could operate based on a single day of data from a mobile computing device 202.

TABLE 1
BEHAVIORAL HEALTH SUMMARIES

| | | | | Reference |
|---|---|---|---|---|
| Column | | Example | | Range (min, |
| Name | Description | Range | | median, max) |
| Behavioral | Behavioral health is made up of core components | (0, | 100) | (13, 84, 99) |
| Health | including social, physical activity, sleep, and routine. | | | |
| Score | This score provides a high-level summary of a | | | |
| | person's overall behavior. | | | |
| Behavioral | This provides a label summary of a person's recent | (−1, | 1) | (−0.991, 0.0, |
| Health | behavioral health. | | | 0.982) |
| Trend | | | | |
| Sleep Score | This is a composite score derived from sleep-related | (0, | 100) | (14, 66, 94) |
| | inferences that provide an overall assessment of | | | |
| | each person's sleep in relation to likely depression. | | | |
| Sleep Score | Provides a label summary of a person's recent sleep | (−1, | 1) | (−0.992, 0.0, |
| Trend | behavior. | | | 0.977) |
| Social | This is a composite score derived from inferences | (0, | 100) | (17, 63, 97) |
| Score | related to individual social behavior. | | | |
| Social | Provides a label summary of a person's recent social | (−1, | 1) | (−0.979, 0.0, |
| Score Trend | behavior. | | | 0.997) |
| Routine | This provides a composite score derived from | (0, | 100) | (14, 82, 98) |
| Score | several underlying routine-related inferences. | | | |
| Routine | Provides a label summary of a person's recent | (−1, | 1) | (−0.987, 0.0, |
| Score Trend | routine. | | | 0.995) |
| Physical | A composite score derived from several measures of | (0, | 100) | (7, 62, 97) |
| Activity | physical activity that provides a high-level summary | | | |
| Score | of a person's physical activity | | | |
| Physical | Provides a label summary of a person's recent | (−1, | 1) | (−0.996, 0.0, |
| Activity | physical activity. | | | 0.999) |
| Trend | | | | |

In an embodiment, each of the Behavioral Health Score, Sleep Score, Social Score, Routine Score, and Physical Activity Score is derived from the inference data, the self-report score values, and the electronic health record in combination with the output of two models, each of which is an ensemble of decision trees that were trained using training data to track a particular item on the PHQ-8 patient health questionnaire for depression. In an embodiment, the training data generally comprises a plurality of records each comprising attribute values for patient demographics and PHQ-8 responses including the value of each response. For example, attribute values in the training data can comprise inferences or behavioral summaries that have been derived from raw sensor data from mobile devices, with labels comprising PHQ-8 responses; records or rows in the training set can correspond to a single patient on a single day. Thus, if a patient provides responses to ten separate PHQs on ten different days, that patient would represent at least ten rows in the training data. In some embodiments, responses can be interpolated to temporally adjacent days. For example, if a patient gives us a PHQ on December 15 and they report a “10”, an embodiment can be programmed to accept that value as representing their mental state not just on the 15th but also on the 14th and 16th.

In one embodiment, the models are trained using the CatBoost open-source machine learning package, which is commercially available at the time of this writing via the domain CATBOOST.AI on the World Wide Web. Each model's parameters were tuned with the training data specified above.

In an embodiment, each model is among the two models comprises a classification model that outputs a probability that, if given a PHQ questionnaire, a patient would give a response above a particular threshold on the item in question. Each individual item model outputs a probability between 0 and 1 inclusive; if prob1 and prob2 are the outputs of the two models used to generate that user's score, the final score is computed as: 100−100*(mean(prob1, prob2)).

While the basic model architecture is the same for each model among the two models, the set of inputs are different for each. For example, the sleep score takes only behavioral signals pertaining to sleep as inputs. There are also separate models trained for males, females, and “default”, which is used in case the patient's gender is unknown, and the specific tracked items and thresholds used depend on the demographic. In one embodiment, the items tracked for each gender and activity category are:

| GENDER/ACTIVITY CATEGORY | ITEM | ITEM |
|---|---|---|
| Routine males | phq2 | phq1 |
| Routine females | phq2 | phq1 |
| Routine default | phq2 | phq1 |
| Overall Behavior males | phq2 | phq1 |
| Overall Behavior females | phq2 | phq1 |
| Overall Behavior default | phq2 | phq1 |
| Activity males | phq3 | phq4 |
| Activity females | phq2 | phq1 |
| Activity default | phq2 | phq1 |
| Sleep males | phq3 | phq4 |
| Sleep females | phq3 | phq4 |
| Sleep default | phq3 | phq4 |
| Social males | phq2 | phq1 |
| Social females | phq2 | phq1 |
| Social default | phq2 | phq1 |

The first row of the table above specifies the use of a first model to track “Routine males-phq2” and a second model to track “Routine males-phq1.” Thus, questionnaire items in the ITEM columns correspond to models. Other rows should be interpreted similarly to specify two models each. Therefore, if a user or patient is male, that user's routine score is computed by aggregating the models “Routine males-phq2” and “Routine males-phq1.” The values “phqX” refer to the Xth item in the 8-question PHQ8 survey. Each of the category-gender combinations specified above can have its own distinct set of inputs to the models.

TABLE 2 illustrates example inferences that can be programmed in various embodiments based on individual sleep behavior. In one embodiment, each of the inferences of TABLE 2 can be calculated when about two days to fourteen days of data has been collected.

TABLE 2
SLEEP BEHAVIOR INFERENCES

| | | | Reference |
|---|---|---|---|
| | | | Range (min, |
| Column Name | Description | Unit/Notes | median, max) |
| sleep_start | This is our estimate for | Follows the ISO 8601 format | NA (see |
| | when sleep started based | including microseconds and the | notes) |
| | on various sensor data on | time zone offset “YYYY-MM- | |
| | the phone. Since this is | DDTHH:MM:SS.mmmmmm + | |
| | based on data from the | HH:MM” | |
| | phone if a user stops | | |
| | interacting with their phone | | |
| | before bed, then this | | |
| | window could be longer | | |
| | than expected. | | |
| sleep_end | This is our estimate for | Follows the ISO 8601 format | NA (see |
| | when sleep ended based | including microseconds and the | notes) |
| | on various sensor data on | time zone offset “YYYY-MM- | |
| | the phone. Since this is | DDTHH:MM:SS.mmmmmm + | |
| | based on data from the | HH:MM” | |
| | phone if a user doesn't | | |
| | interact with their phone | | |
| | when they wake up then | | |
| | this window could be | | |
| | longer than expected. | | |
| sleep_duration | This is the total duration of | seconds | (0, |
| | sleep excluding | | 28080.0, |
| | interruptions. | | 64800.0) |
| sleep_interruptions | This is the number of | Interruptions | (0, 0, 4) |
| | sleep interruption events | | |
| | observed during the night. | | |
| | Only detected if the user | | |
| | interacts with their phone | | |
| | during the interruption. | | |
| chronotype | This is an inferred body | 0 = “extreme early type” | (0, 3, 6) |
| | clock type (i.e., early bird, | 1 = “moderate early type” | |
| | night owl). | 2 = “slight early type” | |
| | | 3 = “neither early nor late type” | |
| | | 4 = “slight late type” | |
| | | 5 = “moderate late type” | |
| | | 6 = extreme late type” | |
| sleep_routine_index | The Sleep Routine Index | (0, 1) | (0.0, 0.87, |
| | (SRI) is a measure that | | 1.0) |
| | assesses the probability | | |
| | that an individual is awake | | |
| | (vs. asleep) at any two | | |
| | time points 24 h apart. A | | |
| | score of 0 implies total | | |
| | random sleep and wake | | |
| | times. A score of 1.0 | | |
| | implies an individual | | |
| | sleeps and wakes at the | | |
| | same time. | | |

In one embodiment, the inference logic 110 is programmed to receive passively collected screen and activity data from among raw sensor data 107 for estimating sleep and wake times as well as sleep interruptions. In one implementation, the inference logic 110 is programmed to merge screen use and activity raw data streams obtained from mobile computing devices 202, correct for any time zone discrepancies, and resample the raw sensor data 107 at five-minute intervals to produce a binary label. For example, the label can be “idle/not idle” to indicate whether the mobile computing device 202 is idle. In an embodiment, the inference logic 110 is programmed to apply a zero-phase filter to the binary label to compute a continuous sleep signal which ranges between 0 and 1 inclusive. When the sleep signal is equal to 1, it indicates a high confidence that the user of the mobile computing device 202 is asleep. In an embodiment, the inference logic 110 is programmed to locate the longest span of time during which the sleep signal is “close” to 1 using empirically defined thresholds and label any periods where the sleep signal is strictly less than 1 as “sleep interruptions”. Given labeled sleep data that has been collected through patient questionnaires, the foregoing method has been determined experimentally to yield highly accurate estimates of sleep and wake times. The piece for estimating interruptions correlates with reported interruptions but may not capture self-reported interruptions where the user did not interact with their mobile computing device 202.

In one embodiment, the inference logic 110 is programmed to receive passively collected screen and activity data from among raw sensor data 107 and the sleep algorithm just described to assign a score based on the user's recent sleep habits. The lower the score, the more of a “morning person” the user is, and vice versa. In an embodiment, inference logic 110 is programmed to receive the passively sensed sleep and wake times from the past six weeks (or whatever data is available if at least one week is available), and for each sleep cycle, the time corresponding to the sleep midpoint is computed. By inspecting the distribution of these midpoints, inference logic 110 is programmed to estimate which sleep cycles corresponded to the user's “off days” (from work or school, for example). In an embodiment, inference logic 110 is programmed to compute the average sleep midpoint of those days off and normalize the results to produce a final score. The score is interpreted as follows:
{score}<1.3-->“Extreme Lark”
1.3<={score}<2.2-->“Moderate Lark”
2.2<={score}<2.8-->“Slight Lark”
2.8<={score}<3.4-->“Average Chronotype”
3.4<={score}<4.0-->“Slight Owl”
4.0<={score}<4.6-->“Moderate Owl”
{score}>=4.6-->“Extreme Owl”
   TABLE 3 illustrates example inferences that can be programmed in various embodiments based on device location. In one embodiment, each of the inferences of TABLE 3 can be calculated when about one to two days of data has been collected. Inferences can include an estimate of a home location and time spent at that home location.

TABLE 3
LOCATION INFERENCES

| | | | Reference |
|---|---|---|---|
| | | | Range (min, |
| Column Name | Description | Unit/Notes | median, max) |
| time_at_home | The time on this | Seconds | (0.0, |
| | date that the user | | 51772.0, |
| | spent within 100 | | 105416.0) |
| | meters of their | | |
| | calculated home | | |
| | location. | | |
| travel_diameter | This is the | Meters | (0.0, |
| | longest distance | | 11899.0, |
| | in meters | | 13496841.0) |
| | between | | |
| | significant | | |
| | locations during | | |
| | the day. | | |
| first_time_leaving_home | This is the first | Follows the ISO 8601 format | NA (see |
| | time a user | including microseconds and the time | notes) |
| | leaves to their | zone offset “YYYY-MM- | |
| | home on a given | DDTHH:MM:SS.mmmmmm + | |
| | day. | HH:MM” | |
| last_time_arriving_home | This is the last | Follows the ISO 8601 format | NA (see |
| | time a user | including microseconds and the time | notes) |
| | arrives at their | zone offset “YYYY-MM- | |
| | home location on | DDTHH:MM:SS.mmmmmm + | |
| | a given day. | HH:MM” | |
| never_left_home | Whether or not | | NA (see |
| | the user spent | | description) |
| | the past 24 hours | | |
| | in their home | | |
| | location. | | |
| time_at_location_{n} | This is the total | Hours | 1: (0.6, |
| | duration that a | | 200.6, |
| | person spends in | | 336.0) |
| | the nth most | | 2: (0.1, |
| | frequent location | | 48.9, 153.8) |
| | (e.g., work) | | 3: (0.1, |
| | | | 10.7, 96.5) |
| | | | 4: (0.1, 4.8, |
| | | | 56.5) |
| | | | 5: (0.1, 3.3, |
| | | | 40.1) |
| mean_time_at_location_{n} | This is the mean | Hours | 1: (0.6, |
| | daily duration that | | 14.64, 24.0) |
| | a person spends | | 2: (0.04, |
| | in the nth most | | 3.71, 10.99) |
| | frequent location | | 3: (0.01, |
| | (e.g., work) | | 0.81, 6.89) |
| | | | 4: (0.01, |
| | | | 0.36, 4.04) |
| | | | 5: (0.01, |
| | | | 0.24, 2.86) |
| num_location_clusters | This is the | Number of locations | (1.0, 26.0, |
| | number of novel | | 197.0) |
| | locations visited | | |
| | by the individual | | |
| loc_entropy | The time- | | (0.0, 1.09, |
| | weighted entropy | | 3.89) |
| | of all novel | | |
| | locations visited | | |
| radius_of_gyration | Root mean | Meters | (0.0, |
| | square (RMS) | | 4182.0, |
| | distance of | | 11069121.0 |
| | location data from | | |
| | the calculated | | |
| | center location of | | |
| | all data gathered | | |
| | on that date. | | |
| timezone_change | True if member | | NA (see |
| | changes time | | description) |
| | zones in each | | |
| | day | | |

In one embodiment, the inference logic 110 is programmed to calculate the loc_entropy or “location entropy” inference noted above as a proxy for how social a user is based on their time at various locations. In one embodiment, the inference logic 110 is programmed to apply a clustering algorithm to identify locations which are most “significant” to the user based on total duration. In one embodiment, the inference logic 110 is programmed to output the mean time spent at the top five locations along with the total number of locations. It also outputs the “location entropy” which is the (non-normalized) entropy of normalized durations at each location.

TABLE 4 illustrates example inferences that can be programmed in various embodiments based on physical activity of an individual. In one embodiment, each of the inferences of TABLE 4 can be calculated when about one to two days of data has been collected.

TABLE 4
PHYSICAL ACTIVITY INFERENCES

| | | | Reference |
|---|---|---|---|
| | | | Range (min, |
| Column Name | Description | Unit/Notes | median, max) |
| active_time | The total time in seconds | Seconds | (0.0, 3120.0. |
| | where users were moving | | 28200.0) |
| | by foot throughout the day. | | |
| activity_percent | Percent of the day that a | Percent, expressed as a | (0.0, 0.036, |
| | user was active. | float from 0 to 1. | 0.3263) |
| step_count | The total steps that | Steps | (1.0, 2779.0, |
| | occurred on this date. | | 35560.0) |
| step_count_mean | mean over a day of number | Steps | (1.0, 29.63, |
| | of steps per bout | | 105.83) |
| step_count_std | std over a day of number of | Steps | (0.0, 27.58, |
| | steps per bout | | 77.51) |
| step_first_time | Timestamp of first recorded | seconds | NA (see |
| | step in a day | | description) |
| step_last_time | Timestamp of last recorded | seconds | NA (see |
| | step in a day | | description) |
| step_floors_down_total | Total number of steps down | steps | (0.0, 8.35, |
| | in a day | | 452.0) |
| step_floors_up_total | Total number of steps up in | steps | (0.0, 8.45, |
| | a day | | 442.0) |
| walking_rate | This is the average number | Steps per minute | (0.5 1.46, |
| | of steps per minute that | | 2.5) |
| | occurred during walking | | |
| | events throughout the day. | | |
| Steps_least_active_5h | A weighted mean of a | Proprietary score meant | (0.0, 3.96, |
| (L5) | user's step counts during | to capture the periodicity | 38.29) |
| | their least active 5 hours of | of a user's activity. | |
| | the day | Irregular weekly physical | |
| | | activity patterns result in a | |
| | | lower score | |
| Steps_most_active_10h | A weighted mean of a | Proprietary score meant | (0.0, 21.18, |
| (M10) | user's step counts during | to capture the periodicity | 156.61) |
| | their most active 10 hours of | of a user's activity. | |
| | the day | Irregular weekly physical | |
| | | activity patterns result in a | |
| | | lower score | |
| steps_relative_amplitude | A relative difference of the | Expressed as a float 0 to | (0.04 0.67, |
| | M10 and L5 inferences | 1. Lower score means the | 1.0) |
| | using the following | most and least active | |
| | calculation: | periods of the day look | |
| | (M10 − L5)/(M10 + L5) | similar for the user | |
| peak_activity_time | Timestamp representing the | | NA (see |
| | middle of the hour with the | | description) |
| | greatest number of steps | | |

In one embodiment, the inference logic 110 is programmed to calculate a passive estimate of walking rate, expressed as steps per second. Many events in the raw pedometer stream in raw sensor data 107, which comes directly from the mobile computing device 202, correspond to short duration movements or longer-duration movements which do not represent walking from point A to point B. In one embodiment, the inference logic 110 is programmed to select the top ten events by distance, check whether a raw activity stream of the raw sensor data 107 labels any part of these events as a “walking” or “unknown” activity (as opposed to “driving” or “cycling” or “running”, for example), then record the start and ending timestamps in an array. In one embodiment, the inference logic 110 is programmed to output the total number of steps between all timestamp pairs in that array divided by the corresponding durations. To address outliers, the output is clipped below at 0.5 steps per second and above at 2.5 steps per second.

In one embodiment, the inference logic 110 is programmed to calculate step rhythmicity values as three metrics which capture the regularity and amplitude of a user's step habits. In one embodiment, the inference logic 110 is programmed to implement the M10, L5, and relative amplitude concepts in EJ van Someren et al., “Circadian rest-activity rhythm disturbances in Alzheimer's disease,” Biol. Psychiatry 1996 Aug. 15; 40(4):259-70, a copy of which is available online at the time of this writing at the HTTP internet path pubmed.ncbi.nlm.nih.gov/8871772, with selected modifications. In an embodiment, a convolution approach is implemented over a longer lookback window and is thus a more robust measure of user activity levels.

In one embodiment, the inference logic 110 is programmed to sample steps taken in ten-minute intervals, and then compute the 5-hour and 10-hour rolling means of these. The output is a continuous “step signal” which the inference logic 110 is programmed to convolve against activity templates which are meant to represent the “ideal” sleep/activity frequency. In one embodiment, the inference logic 110 is programmed to return the maximum value of this convolution for the 10-hour signal and the minimum value for the 5-hour signal in a fixed window of time. The output values are denoted M10 and L5 respectively, although the derivation of the values is different than the values having the same labels in the above-referenced paper. In one embodiment, the inference logic 110 is programmed to compute the relative amplitude as

(M10−L5)/(M10+L5)

In one embodiment, the inference logic 110 is programmed to calculate the activity_percent value based upon a weighted combination of a plurality of raw data values collected from the mobile device. Through experimentation and testing, the inventors have discovered, in an inventive moment, that approximately the first thirty features shown in TABLE 5A are the most useful for calculating the activity_percent value, and that the features shown in TABLE 5B also contribute but to a lesser extent as indicated by their lower weight values.

TABLE 5A
PRIMARY DEVICE ACTIVITY VALUES AND WEIGHTS

| Parameter | Weight |
|---|---|
| steps_relative_amplitude | 7.387098711 |
| step_count_s14 | 6.49239674 |
| steps_relative_amplitude_m10 | 5.854902816 |
| ec_Activity_m14 | 5.72350247 |
| steps_relative_amplitude_m7 | 5.382479397 |
| steps_least_active_5h_s14 | 4.858947883 |
| steps_relative_amplitude_m14 | 4.511826907 |
| ec_PedometerData_m14 | 4.42318498 |
| steps_least_active_5h_s10 | 4.005529847 |
| ec_Activity_s14 | 3.332183355 |
| ec_Activity_m10 | 3.024955309 |
| steps_most_active_10h_m14 | 2.892305542 |
| activity_percent_s14 | 2.496377111 |
| ec_PedometerData_m10 | 2.45977677 |
| ec_Activity_s10 | 2.23398295 |
| steps_most_active_10h_s14 | 2.116410161 |
| step_count_s10 | 2.022993621 |
| ec_Activity_m7 | 1.751991907 |
| steps_least_active_5h_m14 | 1.66805449 |
| ec_PedometerData_s14 | 1.646788015 |
| walking_rate_m14 | 1.485512785 |
| step_count_m14 | 1.37286511 |
| activity_percent_m14 | 1.332140042 |
| total_activity_duration_s14 | 1.196467813 |
| ec_Activity_s7 | 1.158978608 |
| steps_most_active_10h_m10 | 1.144055387 |
| activity_percent_m10 | 1.129205029 |
| ec_PedometerData_m7 | 1.058025009 |
| steps_least_active_5h_s7 | 1.039232313 |
| steps_least_active_5h | 1.011238884 |

TABLE 5B
SECONDARY DEVICE ACTIVITY VALUES AND WEIGHTS

| Parameter | Weight |
|---|---|
| steps_most_active_10h_m7 | 0.94808304 |
| activity_percent_m7 | 0.94465224 |
| walking_rate_s10 | 0.860425202 |
| steps_least_active_5h_m10 | 0.797004896 |
| step_count_s7 | 0.785405886 |
| total_activity_duration_m14 | 0.678043272 |
| step_count_m10 | 0.618685836 |
| steps_least_active_5h_m7 | 0.570419889 |
| ec_PedometerData_s10 | 0.56233687 |
| activity_percent_s10 | 0.544086637 |
| step_count_m7 | 0.530350995 |
| walking_rate_s14 | 0.465600664 |
| walking_rate_m7 | 0.431511197 |
| walking_rate_m10 | 0.425445561 |
| total_activity_duration_m10 | 0.41986661 |
| walking_rate_s7 | 0.397180595 |
| activity_percent_s7 | 0.363468222 |
| steps_most_active_10h_s7 | 0.356992916 |
| total_activity_duration_s10 | 0.312697314 |
| steps_most_active_10h | 0.28752061 |
| steps_most_active_10h_s10 | 0.285533219 |
| walking_rate_t20 | 0.263421863 |
| steps_relative_amplitude_t20 | 0.205068309 |
| steps_relative_amplitude_s14 | 0.170877456 |
| steps_relative_amplitude_s10 | 0.156634279 |
| total_activity_duration_t20 | 0.150356249 |
| step_count_t14 | 0.128387227 |
| steps_least_active_5h_t20 | 0.095973207 |
| steps_relative_amplitude_s7 | 0.091409692 |
| ec_Activity | 0.087207837 |
| total_activity_duration_s7 | 0.084408496 |
| total_activity_duration_m7 | 0.081159434 |
| ec_PedometerData_t20 | 0.080939673 |
| total_activity_duration_t14 | 0.053695812 |
| ec_Activity_t14 | 0.053418873 |
| walking_rate_t14 | 0.048936426 |
| steps_relative_amplitude_t10 | 0.048898432 |
| step_count_t10 | 0.042271607 |
| ec_PedometerData_s7 | 0.033938219 |
| activity_percent_t14 | 0.032571896 |
| steps_least_active_5h_t10 | 0.030593842 |
| activity_percent_t20 | 0.027286286 |
| total_activity_duration | 0.02485071 |
| steps_most_active_10h_t20 | 0.024260971 |
| total_activity_duration_t10 | 0.020751541 |
| steps_most_active_10h_t10 | 0.019508882 |
| activity_percent | 0.017849057 |
| ec_Activity_t20 | 0.016416239 |
| activity_percent_t10 | 0.015702703 |
| step_count | 0.015219026 |
| walking_rate | 0.014710491 |
| ec_Activity_t10 | 0.012669279 |
| walking_rate_t10 | 0.012264321 |
| steps_least_active_5h_t14 | 0.010914042 |
| ec_PedometerData_t14 | 0.009335649 |
| step_count_t20 | 0.007177276 |
| steps_most_active_10h_t14 | 0.0065885 |
| ec_PedometerData | 0.003452499 |
| ec_PedometerData_t10 | 0.001755383 |
| steps_relative_amplitude_t14 | 0.000396683 |

TABLE 6 illustrates example inferences that can be programmed in various embodiments based on mobile computing device activity of an individual.

TABLE 6
DEVICE ACTIVITY INFERENCES

| | | | Reference |
|---|---|---|---|
| | | | Range (min, |
| Column Name | Description | Unit/Notes | median, max) |
| display_events | This is the total number of | events | (1, 265, 1498) |
| | display events that occurred | | |
| | during the day. | | |
| screen_unlocks | This is the total number of | unlock events | (0, 54, 383) |
| | times the device was | | |
| | unlocked during the day. | | |
| device_use_percent | This is the percentage of the | Percent, | (0.0, .196, 1.0) |
| | day that the phone was in | expressed as a | |
| | active use. | float from 0 to 1. | |
| sum_duration_unlock | This is the total duration (in | seconds | (8.6, |
| | seconds) of all unlocked use | | 15895.88, |
| | sessions during the day. | | 86400.0) |
| avg_duration_unlock | This is the average duration | seconds | (5.03, 478.7, |
| | of all unlocked use sessions | | 84807.49) |
| | during the day. | | |
| std_duration_unlock | This is the std of duration of | seconds | (3.11, 755.04, |
| | all unlocked use sessions | | 50567.51) |
| | during the day. | | |
| max_duration_unlock | This is the longest duration | seconds | (5.03, |
| | of unlocked use sessions | | 3825.33, |
| | during the day. | | 84807.49) |
| min_duration_unlock | This is the shortest duration | seconds | (0.0, 16.3, |
| | of unlocked use sessions | | 42902.58) |
| | during the day. | | |
| num_short_unlock | This is the total number of | Short session is | (0.0, 21.12, |
| | short, unlocked usage | defined as less | 247.0) |
| | sessions during the day. | than 30 seconds. | |
| num_long_unlock | This is the total number of | Long session is | (0.0, 1.38, |
| | long unlocked usage | defined as longer | 12.0) |
| | sessions during the day. | than 30 minutes. | |
| burstiness | This is the score evaluating | | (−0.93, 0.36, |
| | the distribution of inter-event | | 1.14) |
| | times of unlock use sessions | | |
| | during the day. | | |
| num_consecutive_use | This is the number of | event | (0.0, 1.06, |
| | consecutive unlock use | | 36.0) |
| | sessions. | | |
| hr_most_unlock | This is the hour when the | Hour timestamp | (0.0, 14.04, |
| | most unlocked event | | 23.0) |
| | happened during the day. | | |
| hr_most_short_unlock | This is the hour when the | Hour timestamp | (0.0, 13.26, |
| | most short, unlocked use | | 23.0) |
| | sessions began during the | | |
| | day. | | |
| hr_longest_unlock | This is the hour when the | Hour timestamp | (0.0, 13.85, |
| | longest duration unlocked | | 23.0) |
| | use session began during | | |
| | the day. | | |
| sum_duration_locked_screen_on | This is the total duration of | seconds | (1.1, 1064.77, |
| | all locked screen-on | | 54032.33) |
| | sessions during the day. | | |
| avg_duration_locked_screen_on | This is the average duration | seconds | (1.1, 18.86, |
| | of all locked screen-on | | 16812.63) |
| | sessions during the day. | | |
| num_locked_screen_on | This is the number of all | event | (1.0, 112.42, |
| | locked screen-on sessions | | 556.0) |
| | during the day. | | |

TABLE 7 illustrates example metadata values that embodiments can calculate or obtain under program control. These metadata fields give general information about the user, the date of the summary, backend systems, the time the summary was calculated, and the mobile computing device of the user.

TABLE 7
METADATA EXAMPLES

| Column Name | Description | Unit/Notes |
|---|---|---|
| user_id | An AWS Cognito ID, serving as | Follows the form “us-east-1:< UUID>” |
| | a unique ID for a specific mobile | where UUID follows the standard UUID |
| | computing device. | format. |
| date | The date for which this inference | Follows the ISO 8601 format for dates |
| | summary was generated. | excluding time.“YYYY-MM-DD” |
| version | This is the version of the | The format here will be “v<Major |
| | inference backend used when | version>.< Minor version>.< Build |
| | this summary was generated. | version>”. For example, v1.0.13 |
| hrcore | This is the version of our core | The format here will be “v<Major |
| | libraries used when generating | version>.< Minor version>.< Build |
| | this summary. | version>”. For example, v1.0.13 |
| app_suspension | Boolean stating whether we | 0 = Not suspended |
| | think the app was suspended | 1 = Suspended |
| | overnight or not. | |
| app_upgrade | Boolean value stating whether | 0 = No app upgrade |
| | the app was upgraded or not on | 1 = App upgraded |
| | this date. | |
| timezone_change | Boolean value stating if the user | 0 = No time zone change |
| | experienced a time zone change | 1 = Time zone change |
| | during the day. | |
| batches | This a comma separated list of | |
| | batch IDs which contained data | |
| | for this date | |
| last_processed_time | This is a timestamp stating the | Follows the ISO 8601 format including |
| | last time this summary was | microseconds and the time zone offset |
| | reprocessed. We reprocess | “YYYY-MM- |
| | summaries whenever new data | DDTHH:MM:SS.mmmmmm + |
| | comes in or when we release a | HH:MM” |
| | new version of the backend. | |

TABLE 8 illustrates example metadata values that embodiments can calculate or obtain under program control. These metadata fields give general information about the user, the date of the summary, backend systems, the time the summary was calculated, and the mobile computing device of the user.

TABLE 8
METADATA EXAMPLES

| | | | Reference |
|---|---|---|---|
| | | | Range (min, |
| Column Name | Description | Unit/Notes | median, max) |
| coverage_activity | This is the percentage of the day that | Percent, | (0.0, .99, 1.99) |
| | we have activity data for the user. | expressed as a | |
| | | float from 0 to 1. | |
| coverage_location | This is the percentage of the day that | Percent, | (0.0, .99, 1.66²) |
| | we have location data for the user. | expressed as a | |
| | | float from 0 to 1. | |
| coverage_display | This is the percentage of the day that | Percent, | (0.0, .99, 1.99²) |
| | we have display data for the user. | expressed as a | |
| | | float from 0 to 1. | |
| coverage | This is the average percentage of the | Percent, | (0.0, .97, |
| | day that we have sensor data for the | expressed as a | 1.59²) |
| | user. | float from 0 to 1. | |
| coverage_code | This is our internal code designating | NA (see | |
| | the likely origin of coverage issues if | description) | |
| | the average coverage was below 70% | | |
| | during the day. | | |
| coverage_explanation | This is a formatted string explaining | NA (see | |
| | our best guess as to why the coverage | description) | |
| | was below 70%. | | |
| overlaps_activity | This is the total number of overlaps | (0, 0, 1220) | (0, 0, 1220) |
| | observed between distinct activity | | |
| | events during the day. | | |
| overlaps_location | This is the total number of overlaps | (0, 0, 170) | (0, 0, 170) |
| | observed between distinct location | | |
| | events during the day. | | |
| overlaps_display | This is the total number of overlaps | (0, 0, 251) | (0, 0, 251) |
| | observed between distinct display | | |
| | events during the day. | | |
| gaps_activity | This is the total number of gaps | (0, 0, 6) | (0, 0, 6) |
| | observed in activity events during the | | |
| | day. | | |
| gaps_location | This is the total number of gaps | (0, 0, 16) | (0, 0, 16) |
| | observed in location events during the | | |
| | day. | | |
| gaps_display | This is the total number of gaps | (0, 0, 18) | (0, 0, 18) |
| | observed in display events during the | | |
| | day. | | |

FIG. 3 illustrates an example of the transformation of inference data into an example digital questionnaire directed to a subject. In the example of FIG. 3, in an embodiment, a questionnaire transformation 240 can be programmed to receive inference data 214 as input and to generate a patient health questionnaire (PHQ) 232 as output. For purposes of illustrating a clear example, FIG. 3 shows an excerpt of a digitally delivered questionnaire based on the PHQ-9 standard, but other embodiments can use different questionnaires or outputs as a substitute for PHQ 232; examples include GAD-7 and Altman Mania.

The questionnaire transformation 240 can be programmed to generate the PHQ 232 dynamically and comprising an association of data values specifying a plurality of different data entry widgets for rendering in a graphical user interface and collecting data through the GUI. The PHQ 232 can be generated to prompt the mobile computing device(s) 202 for input relevant to any of several different health symptoms and/or diagnoses, including mental health symptoms and/or diagnoses. For example, roughly speaking, if the value of the sleep_duration value 216 has a low magnitude, the value of time_at_home value 218 has a high magnitude, and the step_count value 220 has a low magnitude, then the questionnaire transformation 240 can be programmed to generate a PHQ 232 having a plurality of widgets that are associated with questions relevant to a diagnosis of depression. In the example of FIG. 3, the PHQ 232 comprises a plurality of question prompt values 234 each associated with a GUI widget 236, which can be programmed as a pull-down menu, a set of radio buttons, a text entry box, or any of several other GUI widgets.

Further, in an embodiment, inference logic 110 can be programmed to receive an overall summary of core areas of behavior, such as a sleep inference value and social inference value, to dynamically trigger outputting and presenting a patient assessment. As an example, if the inference logic 110 determines that a person's sleep inference value matches a clinical heuristic, such as less than four hours of sleep over two or more days, or a person's sleep is a specified number of hours per day outside of their 30-day mean sleep value, then the inference logic could be programmed to prompt a sleep questionnaire that assesses whether the sleep inference is erroneous and that assesses recent sleep quality of the person's sleep using a clinically validated assessment like the PSQI. Determining whether the sleep inference is erroneous could be implemented by generating questions such as what time the subject went to sleep on the preceding night, for comparison to a stored inference value to determine whether an inference value was calculated incorrectly.

In an embodiment, after the questionnaire transformation 240 generates a PHQ 232, the transformation can be programmed to cause digitally storing the PHQ or transmitting presentation instructions representing the PHQ to the mobile computing device 202. When the PHQ 232 is digitally stored, in one embodiment, the questionnaire transformation 240 can be programmed to initiate a programmed workflow to request one or more online or app-based reviews and approvals of the PHQ. For example, a workflow can be programmed to transmit the PHQ 232, or presentation instructions representing the PHQ, to a healthcare provider or healthcare team that is associated with a patient who uses the mobile computing device 202.

FIG. 4 illustrates an example of the transformation of inference data into digital suggestions that can be created and stored for transmission to a subject.

In the example of FIG. 4, in an embodiment, a suggestion transformation 402 can be programmed to receive inference data 214 as input and to generate a suggestion set 404 comprising one or more digitally stored suggestions 406. The suggestion transformation 402 can be programmed to generate the suggestion set 404 dynamically and comprising an association of data values specifying a plurality of different suggestions 406 for rendering in a graphical user interface and providing notifications, recommendations, or alerts that are relevant to any of several different health symptoms and/or diagnoses, including mental health symptoms and/or diagnoses. For example, in response to identifying a particular combination of values of the sleep duration value 216, time_at_home value 218, and the step_count value 220, or one value, the suggestion transformation 402 can be programmed to generate a suggestion set 404 having a plurality of suggestions that are associated with therapy for a particular category, symptom, condition, or diagnosis.

In the example of FIG. 4, each of the suggestions 406 correlates to a particular value of the sleep_duration value 216 and comprises a plurality of attribute values. Example attributes include a subject ID, a creation time value, a category, a sleep duration value, a count of sleep interruptions, and a suggestion text. The attribute values can be digitally stored in a database record, such as a row of a table, with a row identifier or suggestion identifier serving as a key value. In some embodiments, each of the suggestions 406 correlates to a different value of sleep duration, as seen in FIG. 4. In other embodiments, attributes other than the sleep duration value can be used, and suggestions can relate to issues other than sleep. In some embodiments, one or more of the suggestions 406 can be pre-populated in a message that is digitally stored in the health analysis application 108 or storage 104, so clinical staff can trigger the transmission of the message to a patient via HIPAA-compliant text messaging, an in-app notification, or face-to-face. Suggestions 406 can be structured as intervention or pre-intervention checklists.

In an embodiment, the inference logic 110 is programmed to analyze all data inferred from all the mobile devices and to classify or stratify subjects into risk categories. Generally, risk stratification logic 506 can be programmed to receive inference data 214 as input, to execute one or more programmed risk stratification rules or tests, and output via one or more output paths 520, 522 one or more risk values selected from among a plurality of risk values 512. For example, execution or risk stratification logic for the inference data 214 that is associated with a particular subject could result in selecting an Urgent risk value 514 from the risk values 512.

Furthermore, in an embodiment, the risk stratification logic 506 can be programmed to execute a write operation 530 to write the Urgent risk value 514, or another risk value that was selected, to a subject record that is rendered or presented in a care team interface 532. In an embodiment, the care team interface is displayed by the health analysis application 108 generating one or more dynamic HTML pages that the healthcare provider computer 130 can render and display using a browser. Alternatively, healthcare provider computer 130 can execute a mobile application or app that is compatible with health analysis application 108. The subject record and the care team interface 532 can be configured to identify a particular subject or patient and to display the risk value 514 in a risk report panel 534, optionally with one or more other metrics or values relating to subject risk or subject condition. The care team interface 532 can include a GUI widget that is programmed to receive input to communicate an acknowledgment that a care team member saw or reviewed the risk report panel 534.

2.3 User Interface Examples

FIG. 5 illustrates an example data flow in which specified data sources are transformed into a strata of risk values to update a clinical dashboard. In the example of FIG. 5, the plurality of risk values 512 comprises four (4) risk values denoted Emergent, Urgent, Medium, and Low; however, other embodiments can use different numbers and values to stratify risk of subjects. Further, in the example of FIG. 5, the risk stratification logic 506 can be programmed to receive and act upon input other than inference data 214, such as a patient BER 502, and/or one or more self-reported score values that the subject has entered using mobile computing device 202. The programmed techniques that have been described in connection with FIG. 2, FIG. 3, FIG. 4, FIG. 5 can form parts of an end-to-end data processing and response workflow that integrates a feedback loop.

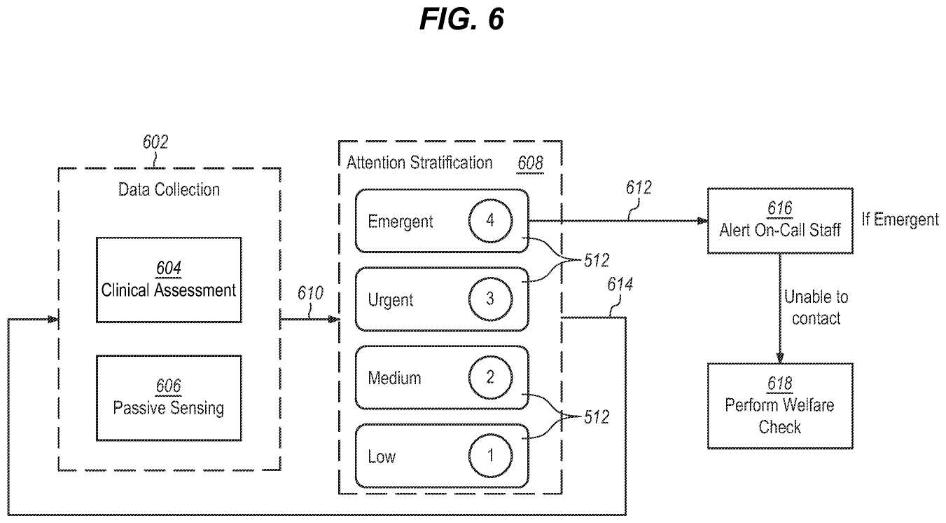

FIG. 6 illustrates an example of a workflow that is programmed to collect data, stratify the attention of a care team concerning a plurality of subjects, and initiate responses using other systems. The example of FIG. 6 generally comprises a data collection stage 602 coupled to an attention stratification stage 608, which is coupled to an alert operation 616, which optionally can initiate a welfare operation 618. The attention stratification stage 608 also has a feedback loop 614 that is coupled to the data collection stage 602.

In an embodiment, the data collection stage 602 comprises one or more of a clinical assessment 604 and passive sensing 606. The clinical assessment 604 can comprise a traditional clinical assessment of a subject in person or using teleconference or virtual meeting technology, after which a clinician stores one or more digital data values in an electronic health record of a subject. The passive sensing 606 can comprise the collection of data values passively from a mobile computing device of the subject in the manner that has been previously described, including execution of transformation operations to create and store data values representing inferences from the raw data values that were collected.

Path 610 represents programmatically transferring the data collected at data collection stage 602 to the attention stratification stage 608, which is programmed to analyze data from the data collection stage and to output a particular risk value, on path 612, that has been selected from among a plurality of available risk values 512. In an embodiment, path 612 comprises a programmatic call to the alert operation 616. In an embodiment, the alert operation 616 is programmed to update the care team interface 532 (FIG. 5), and to determine whether the particular risk value is equal to Emergent or a functionally similar risk value. In response to determining that the particular risk value is equal to Emergent or a functionally similar risk value, the alert operation 616 is programmed to initiate an alert communication to one or more on-call members of staff or to care team members. The alert operation 616 can be programmed to initiate an alert communication by executing one or more programmatic calls to APIs or other services to initiate a voice-over-internet-protocol (VoIP) call to a cellular telephone number or soft phone number of the one or more on-call members of staff or care team members, to call a text messaging service to initiate one or more text messages to the one or more on-call members of staff or care team members, or to call an application server to result in generating an in-app message at a compatible mobile device application running on a mobile computing device of the one or more on-call members of staff or care team members.

Typically, executing the alert operation 616 induces the one or more on-call members of staff or care team members to contact the subject by a cellular radiotelephone call, text message, or email message to a known number or address of the subject. If the subject cannot be contacted, then the workflow may transition to welfare operation 618 in which the one or more on-call members of staff or care team members initiate or personally perform an in-person welfare check of the subject.

Using path 614, the attention stratification stage 608 can be programmed to transmit the particular risk value that the stage selected to the data collection stage 602. For example, a risk value 512 of “Urgent” could be sent via path 614, using an API call or other programmatic means, to the passive sensing 606 to form an additional data item for use in inference transformations. Or the risk value 512 could be sent via path 614 to an EHR or other record that was created and stored using clinical assessment 604.

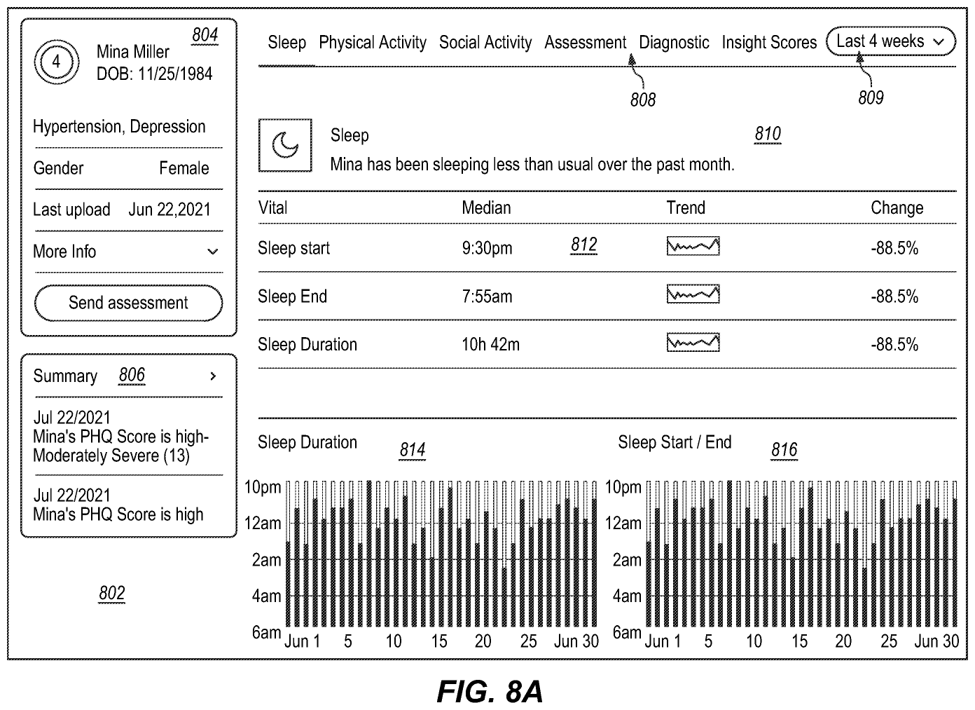

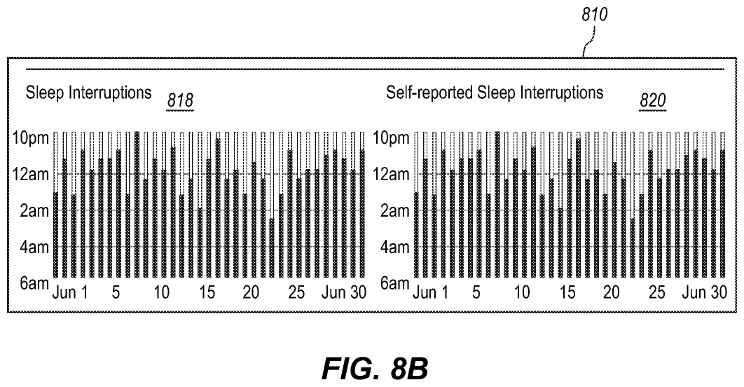

FIG. 8A, FIG. 8B illustrate portions of an example of a care team interface, in one embodiment. Referring first to FIG. 8A, in one embodiment, the health analysis application 108 (FIG. 1A) can be programmed to generate presentation instructions which, when transmitted via network 106 to healthcare provider computer 130 and rendered at the healthcare provider computer, cause displaying a care team interface 802 having a patient data panel 804, summary panel 806, toolbar 808 and data window 810. The patient data panel 804 can be programmed to show basic patient identifying data such as name, date of birth, diagnoses, gender, last update date, and so forth. In some embodiments, depending on permissions or access controls applicable to a user, data in patient data panel can be partly or fully anonymized.

In an embodiment, the summary panel 806 can be programmed to display an expansion link and one or more notifications or alerts pertaining to the patient. In the example of FIG. 8A, an expansion link is denoted using a graphical icon “>” and which when selected is programmed to expand panel 806 and present a more detailed review of alerts or notifications. In an embodiment, the toolbar 808 is programmed to display a plurality of named links which when selected cause displaying the data window 810 in different forms or configurations and with different data. Examples of named links include SLEEP, PHYSICAL ACTIVITY, SOCIAL ACTIVITY, ASSESSMENT, DIAGNOSTIC, INSIGHT SCORES.

In the example of FIG. 8A, the SLEEP link has been selected and the data window 810 shows data relating to the patient's sleep activity, based on the passively collected data and inferences that have been previously described in other sections. In one embodiment, data window 810 for SLEEP data comprises a vital signs table 812, a sleep duration histogram 814 and a sleep time histogram 816. Referring to FIG. 8B, data window 810 also can comprise a sleep interruption histogram 818 and self-reported sleep interruption histogram 820. The vital signs table 812 can provide a summary of sleep-related data based on the passively collected data and inferences that have been previously described in other sections. For example, vital signs table 812 can be programmed to display rows specifying vital signs such as sleep start, end, and duration; each row can comprise multiple column attributes such as median, trend, and change. The health analysis application 108 (FIG. 1A) can be programmed to calculate values for the column attributes and/or to generate and transmit one or more database queries to database 104 to cause retrieving relevant data and calculating the column attribute values. For each of the sleep duration histogram 814, sleep time histogram 816, sleep interruption histogram 818, and self-reported sleep interruption histogram 820, health analysis application 108 (FIG. 1A) can be programmed to transmit one or more database queries to database 104 to cause retrieving relevant data to form histogram bars and use a presentation library or graphics library to generate presentation instructions that package the data for rendering at the healthcare provider computer 130.

In an embodiment, care team interface 802 also can comprise a scope widget 809 programmed as a pull-down menu specifying a plurality of different time windows or intervals. In an embodiment, user input from the healthcare provider computer 130 can select the scope widget 809 and a different option from the pull-down menu. The health analysis application 108 can be programmed, in response to a selection of the widget 809 specifying a different option, to generate one or more new queries to database 104 and/or conduct one or more new calculations based on the selected option, thereby generating new column attribute values and histogram bars for vital signs table 812, sleep duration histogram 814, sleep time histogram 816, sleep interruption histogram 818 and self-reported sleep interruption histogram 820, and to transmit updated presentation instructions to the healthcare provider computer 130 to cause updating the care team interface 808 in real-time in response to the selection.

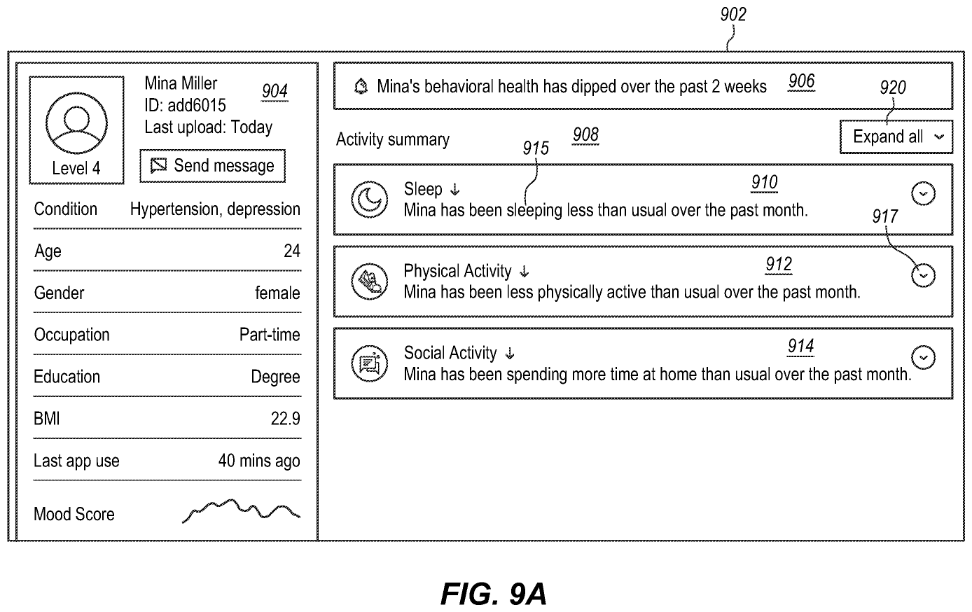

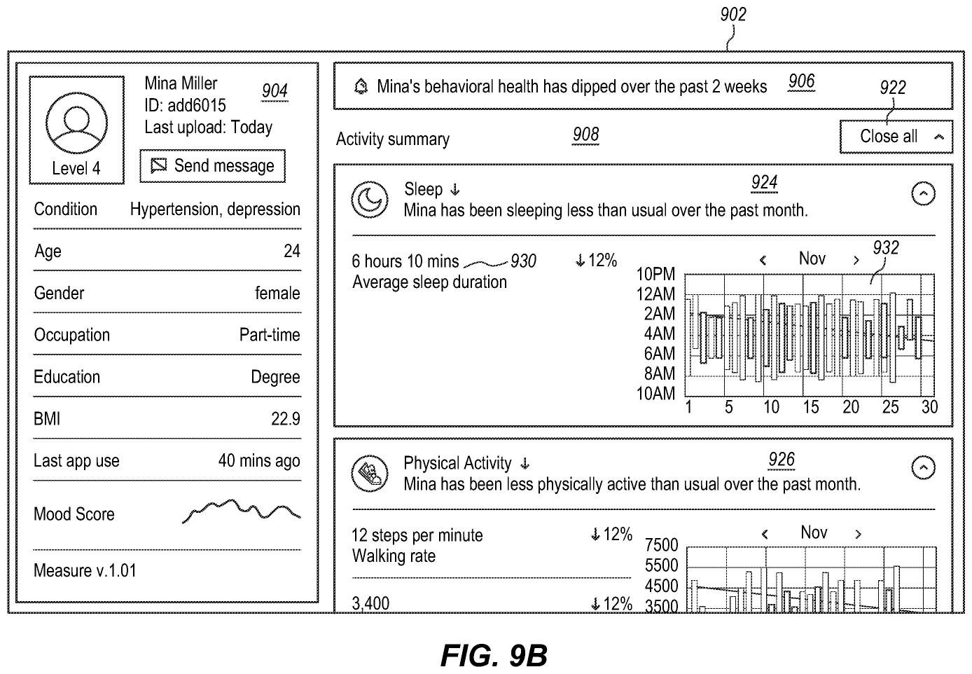

FIG. 9A illustrates a portion of an example of a care team interface in which certain graphical panels are collapsed. FIG. 9B illustrates a portion of an example of a care team interface in which certain graphical panels are expanded. Referring first to FIG. 9A, in one embodiment, the health analysis application 108 (FIG. 1A) can be programmed to generate presentation instructions which, when transmitted via network 106 to healthcare provider computer 130 and rendered at the healthcare provider computer, cause displaying a care team interface 902 having a patient data panel 904, key alert panel 906, activity summary panel 908, and all-expansion control 920. The patient data panel 904 can be programmed in the same manner as described herein for panel 804 (FIG. 8A). The Referring now to FIG. 9B, in an embodiment, health analysis application 108 (FIG. 1A) can be programmed to generate a specific alert or notification for the key alert panel 906 based on applying programmed rules or heuristics that detect changes in the behavioral health score value of TABLE 1. For example, one or more changes in magnitude of the score value within pairs of specified thresholds can be associated with different programmed text messages. A specific alert or notification for the key alert panel 906 can be determined as follows. In response to user input from the healthcare provider computer 130 that opens the care team interface 902, health analysis application 108 can be programmed to retrieve a previously stored behavioral health score value for a prior period, to calculate an updated, current behavioral health score value in the manner previously described for the metrics that contribute to TABLE 1, to compare the two values, and to select the specific text message based upon the prior value and the current value matching one or more threshold values.

In an embodiment, activity summary panel 908 is programmed to display a plurality of activity panels 910, 912, 914, each being associated with a different patient activity, such as sleep, physical activity, and social activity. Each of the activity panels 910, 912, 914 can be presented initially in a collapsed state as shown in FIG. 9A and can include a notification or alert 915. Each notification or alert 915 for a particular activity panel 910, 912, 914 can be calculated in real-time in response to opening the care team interface 902 based on the metrics shown herein in the TABLEs and a plurality of different programmed rules and statically stored text statements.

Each of the activity panels 910, 912, 914 can be programmed to display an expansion widget 917 in the panel. In an embodiment, user input from the healthcare provider computer 130 to select an expansion widget 917 of a particular panel causes health analysis application 108 to generate and transmit updated presentation instructions to render the panel in an expanded state. Or, in an embodiment, user input from the healthcare provider computer 130 to select the all-expansion control 920 causes health analysis application 108 to generate and transmit updated presentation instructions to render each of the activity panels 910, 912, 914 in an expanded state.

Referring now to FIG. 9B, when all the activity panels 910, 912, 914 are in an expanded state, each of the panels can be programmed to display different messages, individual metrics, trends, and graphics. For example, FIG. 9B shows a sleep panel 924 and a physical activity panel 926 in expanded states, corresponding to activity panels 910, 912 of FIG. 9A. The sleep panel 924 can be programmed to display an average sleep duration 930 as a numeric value with a label or message, and a bar chart 932 illustrating sleep duration over a specified period. The physical activity panel 926 can be programmed to show steps per minute or other walking rate metrics and a bar chart presenting that metric over the same period as in panel 924, or a different period.

2.4 Benefits and Improvements of Embodiments

Embodiments can achieve the following technical, clinical and care benefits. The technical improvement is to present a succinct actionable summary of an individual patient's behavioral health within the EHR or clinical view that involves collection of sensor data, collection of available behavioral inferences via wearables, operation systems etc., and the aggregation and homogenization of this information & transformation into clinical information view.

The clinical improvements include 1. Objective measurement of behavior in several domains that are highly germane to the assessment of psychiatric illness, with relevance to the functional impact of illness; 2. Continuous measurement of those domains; 3. Ecologically valid measurement of those domains. This information has previously only been assessed via clinical interview, which suffers from subjectivity, lack of continuity, and the unreasonable reliance on patient memory in the context of disorders that impact cognitive capacities such as memory.

This information enables, for the first time, continuous monitoring of patient mental health status and degree of functional impairment, providing clinicians and patients with actionable data regarding the impact of the disorder and interventions, including side effects. For example, a depressed patient undergoing treatment may endorse improved mood from two separate interventions with differing effects on functioning, providing the clinician with crucial information to consider when deciding between interventions.

Care benefits include more efficient care (because treatments are more targeted and treatment efficacy can be assessed more rapidly thereby reducing negative outcomes duration). Behavioral health vital signs can also be used to provide patients with individual summaries of their behavioral health to 1) increase personal awareness, 2) provide positive feedback on behavioral improvements (e.g., you've been sleeping a little more), 3) drive educational content relevant to the area of need (e.g., loneliness via social isolation)

This data helps to empower patients by providing feedback on the impact of different interventions and behavioral choices. Personal awareness and knowledge can help influence patients to make positive behavioral health changes.

### 3. Implementation Example—Hardware Overview

According to one embodiment, the techniques described herein are implemented by at least one computing device. The techniques may be implemented in whole or in part using a combination of at least one server computer and/or other computing devices that are coupled using a network, such as a packet data network. The computing devices may be hard-wired to perform the techniques or may include digital electronic devices such as at least one application-specific integrated circuit (ASIC) or field programmable gate array (FPGA) that is persistently programmed to perform the techniques or may include at least one general purpose hardware processor programmed to perform the techniques pursuant to program instructions in firmware, memory, other storage, or a combination. Such computing devices may also combine custom hard-wired logic, ASICs, or FPGAs with custom programming to accomplish the described techniques. The computing devices may be server computers, workstations, personal computers, portable computer systems, handheld devices, mobile computing devices, wearable devices, body mounted or implantable devices, smartphones, smart appliances, internetworking devices, autonomous or semi-autonomous devices such as robots or unmanned ground or aerial vehicles, any other electronic device that incorporates hard-wired and/or program logic to implement the described techniques, one or more virtual computing machines or instances in a data center, and/or a network of server computers and/or personal computers.

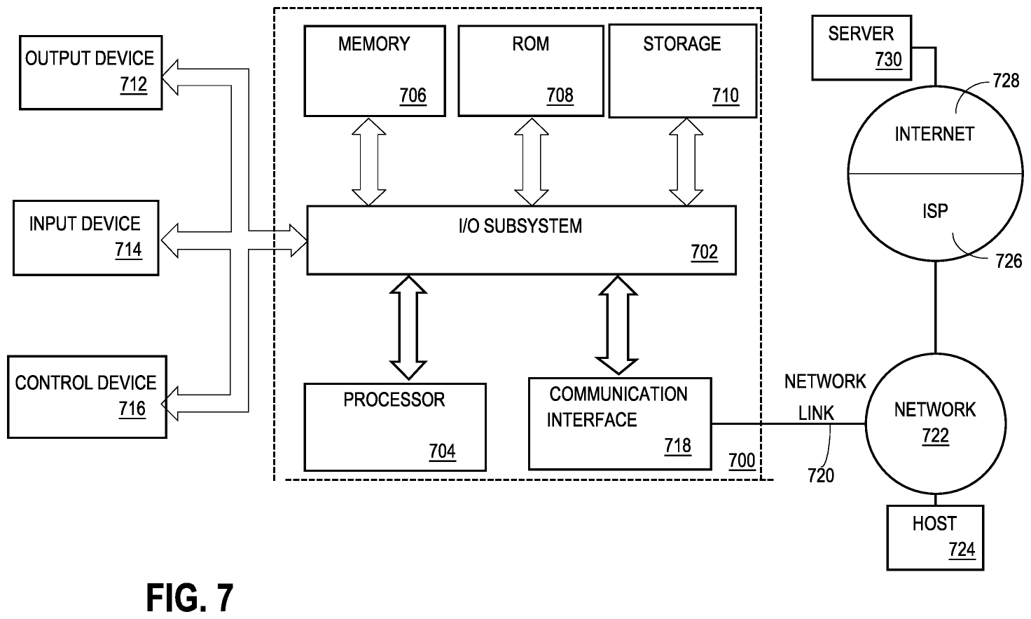

FIG. 7 is a block diagram that illustrates an example computer system with which an embodiment may be implemented. In the example of FIG. 7, a computer system 700 and instructions for implementing the disclosed technologies in hardware, software, or a combination of hardware and software, are represented schematically, for example as boxes and circles, at the same level of detail that is commonly used by persons of ordinary skill in the art to which this disclosure pertains for communicating about computer architecture and computer systems implementations.

Computer system 700 includes an input/output (I/O) subsystem 702 which may include a bus and/or other communication mechanism(s) for communicating information and/or instructions between the components of the computer system 700 over electronic signal paths. The I/O subsystem 702 may include an I/O controller, a memory controller and at least one I/O port. The electronic signal paths are represented schematically in the drawings, for example as lines, unidirectional arrows, or bidirectional arrows.

At least one hardware processor 704 is coupled to I/O subsystem 702 for processing information and instructions. Hardware processor 704 may include, for example, a general-purpose microprocessor or microcontroller and/or a special-purpose microprocessor such as an embedded system or a graphics processing unit (GPU) or a digital signal processor or ARM processor. Processor 704 may comprise an integrated arithmetic logic unit (ALU) or may be coupled to a separate ALU.

Computer system 700 includes one or more units of memory 706, such as a main memory, which is coupled to I/O subsystem 702 for electronically digitally storing data and instructions to be executed by processor 704. Memory 706 may include volatile memory such as various forms of random-access memory (RAM) or other dynamic storage device. Memory 706 also may be used for storing temporary variables or other intermediate information during execution of instructions to be executed by processor 704. Such instructions, when stored in non-transitory computer-readable storage media accessible to processor 704, can render computer system 700 into a special-purpose machine that is customized to perform the operations specified in the instructions.

Computer system 700 further includes non-volatile memory such as read only memory (ROM) 708 or other static storage device coupled to I/O subsystem 702 for storing information and instructions for processor 704. The ROM 708 may include various forms of programmable ROM (PROM) such as erasable PROM (EPROM) or electrically erasable PROM (EEPROM). A unit of persistent storage 710 may include various forms of non-volatile RAM (NVRAM), such as FLASH memory, or solid-state storage, magnetic disk, or optical disk such as CD-ROM or DVD-ROM and may be coupled to I/O subsystem 702 for storing information and instructions. Storage 710 is an example of a non-transitory computer-readable medium that may be used to store instructions and data which when executed by the processor 704 cause performing computer-implemented methods to execute the techniques herein.

The instructions in memory 706, ROM 708 or storage 710 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. The instructions may implement a web server, web application server or web client. The instructions may be organized as a presentation layer, application layer and data storage layer such as a relational database system using structured query language (SQL) or no SQL, an object store, a graph database, a flat file system or other data storage.

Computer system 700 may be coupled via I/O subsystem 702 to at least one output device 712. In one embodiment, output device 712 is a digital computer display. Examples of a display that may be used in various embodiments include a touch screen display or a light-emitting diode (LED) display or a liquid crystal display (LCD) or an e-paper display. Computer system 700 may include other type(s) of output devices 712, alternatively or in addition to a display device. Examples of other output devices 712 include printers, ticket printers, plotters, projectors, sound cards or video cards, speakers, buzzers or piezoelectric devices or other audible devices, lamps or LED or LCD indicators, haptic devices, actuators, or servos.

At least one input device 714 is coupled to I/O subsystem 702 for communicating signals, data, command selections or gestures to processor 704. Examples of input devices 714 include touch screens, microphones, still and video digital cameras, alphanumeric and other keys, keypads, keyboards, graphics tablets, image scanners, joysticks, clocks, switches, buttons, dials, slides, and/or various types of sensors such as force sensors, motion sensors, heat sensors, accelerometers, gyroscopes, and inertial measurement unit (INU) sensors and/or various types of transceivers such as wireless, such as cellular or Wi-Fi, radio frequency (RF) or infrared (IR) transceivers and Global Positioning System (GPS) transceivers.

Another type of input device is a control device 716, which may perform cursor control or other automated control functions such as navigation in a graphical interface on a display screen, alternatively or in addition to input functions. Control device 716 may be a touchpad, a mouse, a trackball, or cursor direction keys for communicating direction information and command selections to processor 704 and for controlling cursor movement on display 712. The input device may have at least two degrees of freedom in two axes, a first axis (e.g., x) and a second axis (e.g., y), that allows the device to specify positions in a plane. Another type of input device is a wired, wireless, or optical control device such as a joystick, wand, console, steering wheel, pedal, gearshift mechanism or other type of control device. An input device 714 may include a combination of multiple different input devices, such as a video camera and a depth sensor.

In another embodiment, computer system 700 may comprise an internet of things (IoT) device in which one or more of the output device 712, input device 714, and control device 716 are omitted. Or, in such an embodiment, the input device 714 may comprise one or more cameras, motion detectors, thermometers, microphones, seismic detectors, other sensors or detectors, measurement devices or encoders and the output device 712 may comprise a special-purpose display such as a single-line LED or LCD display, one or more indicators, a display panel, a meter, a valve, a solenoid, an actuator or a servo.

When computer system 700 is a mobile computing device, input device 714 may comprise a global positioning system (GPS) receiver coupled to a GPS module that is capable of triangulating to a plurality of GPS satellites, determining and generating geo-location or position data such as latitude-longitude values for a geophysical location of the computer system 700. Output device 712 may include hardware, software, firmware, and interfaces for generating position reporting packets, notifications, pulse or heartbeat signals, or other recurring data transmissions that specify a position of the computer system 700, alone or in combination with other application-specific data, directed toward host 724 or server 730.

Computer system 700 may implement the techniques described herein using customized hard-wired logic, at least one ASIC or FPGA, firmware and/or program instructions or logic which when loaded and used or executed in combination with the computer system causes or programs the computer system to operate as a special-purpose machine. According to one embodiment, the techniques herein are performed by computer system 700 in response to processor 704 executing at least one sequence of at least one instruction contained in main memory 706. Such instructions may be read into main memory 706 from another storage medium, such as storage 710. Execution of the sequences of instructions contained in main memory 706 causes processor 704 to perform the process steps described herein. In alternative embodiments, hard-wired circuitry may be used in place of or in combination with software instructions.

The term “storage media” as used herein refers to any non-transitory media that store data and/or instructions that cause a machine to operate in a specific fashion. Such storage media may comprise non-volatile media and/or volatile media. Non-volatile media includes, for example, optical or magnetic disks, such as storage 710. Volatile media includes dynamic memory, such as memory 706. Common forms of storage media include, for example, a hard disk, solid state drive, flash drive, magnetic data storage medium, any optical or physical data storage medium, memory chip, or the like.

Storage media is distinct from but may be used in conjunction with transmission media. Transmission media participates in transferring information between storage media. For example, transmission media includes coaxial cables, copper wire and fiber optics, including the wires that comprise a bus of I/O subsystem 702. Transmission media can also take the form of acoustic or light waves, such as those generated during radio-wave and infra-red data communications.

Various forms of media may be involved in carrying at least one sequence of at least one instruction to processor 704 for execution. For example, the instructions may initially be carried on a magnetic disk or solid-state drive of a remote computer. The remote computer can load the instructions into its dynamic memory and send the instructions over a communication link such as a fiber optic or coaxial cable or telephone line using a modem. A modem or router local to computer system 700 can receive the data on the communication link and convert the data to a format that can be read by computer system 700. For instance, a receiver such as a radio frequency antenna or an infrared detector can receive the data carried in a wireless or optical signal and appropriate circuitry can provide the data to I/O subsystem 702 such as place the data on a bus. I/O subsystem 702 carries the data to memory 706, from which processor 704 retrieves and executes the instructions. The instructions received by memory 706 may optionally be stored on storage 710 either before or after execution by processor 704.

Computer system 700 also includes a communication interface 718 coupled to bus 702. Communication interface 718 provides a two-way data communication coupling to network link(s) 720 that are directly or indirectly connected to at least one communication network, such as a network 722 or a public or private cloud on the Internet. For example, communication interface 718 may be an Ethernet networking interface, integrated-services digital network (ISDN) card, cable modem, satellite modem, or a modem to provide a data communication connection to a corresponding type of communications line, for example an Ethernet cable or a metal cable of any kind or a fiber-optic line or a telephone line. Network 722 broadly represents a local area network (LAN), wide-area network (WAN), campus network, internetwork, or any combination thereof. Communication interface 718 may comprise a LAN card to provide a data communication connection to a compatible LAN, or a cellular radiotelephone interface that is wired to send or receive cellular data according to cellular radiotelephone wireless networking standards, or a satellite radio interface that is wired to send or receive digital data according to satellite wireless networking standards. In any such implementation, communication interface 718 sends and receives electrical, electromagnetic, or optical signals over signal paths that carry digital data streams representing various types of information.

Network link 720 typically provides electrical, electromagnetic, or optical data communication directly or through at least one network to other data devices, using, for example, satellite, cellular, Wi-Fi, or BLUETOOTH technology. For example, network link 720 may provide a connection through a network 722 to a host computer 724.

Furthermore, network link 720 may provide a connection through network 722 or to other computing devices via internetworking devices and/or computers that are operated by an Internet Service Provider (ISP) 726. ISP 726 provides data communication services through a world-wide packet data communication network represented as internet 728. A server computer 730 may be coupled to internet 728. Server 730 broadly represents any computer, data center, virtual machine, or virtual computing instance with or without a hypervisor, or computer executing a containerized program system such as DOCKER or KUBERNETES. Server 730 may represent an electronic digital service that is implemented using more than one computer or instance and that is accessed and used by transmitting web services requests, uniform resource locator (URL) strings with parameters in HTTP payloads, API calls, app services calls, or other service calls. Computer system 700 and server 730 may form elements of a distributed computing system that includes other computers, a processing cluster, server farm or other organization of computers that cooperate to perform tasks or execute applications or services. Server 730 may comprise one or more sets of instructions that are organized as modules, methods, objects, functions, routines, or calls. The instructions may be organized as one or more computer programs, operating system services, or application programs including mobile apps. The instructions may comprise an operating system and/or system software; one or more libraries to support multimedia, programming or other functions; data protocol instructions or stacks to implement TCP/IP, HTTP or other communication protocols; file format processing instructions to parse or render files coded using HTML, XML, JPEG, MPEG or PNG; user interface instructions to render or interpret commands for a graphical user interface (GUI), command-line interface or text user interface; application software such as an office suite, internet access applications, design and manufacturing applications, graphics applications, audio applications, software engineering applications, educational applications, games or miscellaneous applications. Server 730 may comprise a web application server that hosts a presentation layer, application layer and data storage layer such as a relational database system using structured query language (SQL) or no SQL, an object store, a graph database, a flat file system or other data storage.

Computer system 700 can send messages and receive data and instructions, including program code, through the network(s), network link 720 and communication interface 718. In the Internet example, a server 730 might transmit a requested code for an application program through Internet 728, ISP 726, local network 722 and communication interface 718. The received code may be executed by processor 704 as it is received, and/or stored in storage 710, or other non-volatile storage for later execution.

The execution of instructions as described in this section may implement a process in the form of an instance of a computer program that is being executed and consisting of program code and its current activity. Depending on the operating system (OS), a process may be made up of multiple threads of execution that execute instructions concurrently. In this context, a computer program is a passive collection of instructions, while a process may be the actual execution of those instructions. Several processes may be associated with the same program; for example, opening several instances of the same program often means more than one process is being executed. Multitasking may be implemented to allow multiple processes to share processor 704. While each processor 704 or core of the processor executes a single task at a time, computer system 700 may be programmed to implement multitasking to allow each processor to switch between tasks that are being executed without having to wait for each task to finish. In an embodiment, switches may be performed when tasks perform input/output operations, when a task indicates that it can be switched, or on hardware interrupts. Time-sharing may be implemented to allow fast response for interactive user applications by rapidly performing context switches to provide the appearance of concurrent execution of multiple processes simultaneously. In an embodiment, for security and reliability, an operating system may prevent direct communication between independent processes, providing strictly mediated and controlled inter-process communication functionality.

In the foregoing specification, embodiments of the invention have been described with reference to numerous specific details that may vary from implementation to implementation. The specification and drawings are, accordingly, to be regarded in an illustrative rather than a restrictive sense. The sole and exclusive indicator of the scope of the invention, and what is intended by the applicants to be the scope of the invention, is the literal and equivalent scope of the set of claims that issue from this application, in the specific form in which such claims issue, including any subsequent correction.

What is claimed is:

1. A computer-implemented method comprising:
receiving at a server computer via a data communication network, a plurality of raw sensor data and a plurality of self-report score values from a plurality of mobile computing devices;
using the server computer, for a particular mobile computing device from among the plurality of mobile computing devices; calculating a plurality of inference data based upon transformations of the raw sensor data and the plurality of self-report score values;
the server computer accessing via the network a patient electronic health record that is associated with a user of the particular mobile computing device;
inputting the raw sensor data and the electronic health record to a plurality of trained machine learning models;
operating the plurality of trained machine learning models in an inference phase to generate outputs specifying probabilities that a user of the particular mobile computing device would respond above a particular threshold for each item of the patient health questionnaire;
based on the probabilities, the server computer calculating a risk value of the user, updating a graphical user interface of a healthcare provider computer to display the risk value, and based on the risk value, and transmitting one or more of an on-call staff alert or a welfare check request.

2. The computer-implemented method of claim 1, the inference data comprising one or more of a Behavioral Health Score, Sleep Score, Social Score, Routine Score, and Physical Activity Score.

3. The computer-implemented method of claim 1, the plurality of trained machine learning models each comprising an ensemble of decision trees that have been trained using training data to track a particular item on a patient health questionnaire for depression.

4. The computer-implemented method of claim 3, the training data comprising a plurality of records each comprising attribute values for patient demographics and one or more inferences or behavioral summaries that have been derived from the raw sensor data, and one or more sets of responses to items of the patient health questionnaire.

5. The computer-implemented method of claim 4, the training data comprising, for a particular patient that is associated with the particular mobile computing device, a plurality of sets of responses to the patient health questionnaire corresponding to a plurality of different days.

6. The computer-implemented method of claim 4, the training data comprising, for a particular patient that is associated with the particular mobile computing device, a plurality of sets of responses to the PHQ-8 patient health questionnaire for depression and corresponding to a plurality of different days.

7. The computer-implemented method of claim 3, further comprising, based on the inference data, selecting a digitally stored different questionnaire, transmitting the different questionnaire to the particular mobile computing device, receiving a plurality of responses corresponding to plural items in the different questionnaire, and updating the training data based on the responses.

8. The computer-implemented method of claim 1, each model among the plurality of trained machine learning models comprising a classification model that is associated with a particular item of the patient health questionnaire, each classification model being configured to output a probability that, if given the patient health questionnaire, a response of a patient would exceed a particular threshold value for the particular item.

9. The computer-implemented method of claim 8, the plurality of trained machine learning models comprising pairs of models, each of the pairs being associated with a common category of gender and activity, each of the models being trained on a particular item of the PHQ-8 patient health questionnaire for depression.

10. The computer-implemented method of claim 9, the particular item comprising any of item “1”, “2”, “3”, or “4” of the PHQ-8 patient health questionnaire for depression.

11. The computer-implemented method of claim 1, further comprising, based on the inference data, selecting and transmitting to the particular mobile computing device a plurality of suggestion messages each comprising a text suggestion for presentation on the particular mobile computing device.

12. The computer-implemented method of claim 1, the plurality of raw sensor data comprising location data, pedometer data, activity data, and device data, the device data specifying any of device unlock events, device lock events, device lock screen display events, application launch events, application dismiss events.

13. The computer-implemented method of claim 1, the activity data specifying physical interactions of a user with the particular mobile computing device including one or more gestures, taps, operation of hardware switches or controls.

14. One or more non-transitory computer-readable data storage media storing one or more sequences of instructions which when executed using one or more processors cause the one or more processors to execute:
receiving at a server computer via a data communication network, a plurality of raw sensor data and a plurality of self-report score values from a plurality of mobile computing devices;
using the server computer, for a particular mobile computing device from among the plurality of mobile computing devices; calculating a plurality of inference data based upon transformations of the raw sensor data and the plurality of self-report score values;
the server computer accessing via the network a patient electronic health record that is associated with a user of the particular mobile computing device;
inputting the raw sensor data and the electronic health record to a plurality of trained machine learning models;
operating the plurality of trained machine learning models in an inference phase to generate outputs specifying probabilities that a user of the particular mobile computing device would respond above a particular threshold for each item of the patient health questionnaire;
based on the probabilities, the server computer calculating a risk value of the user, updating a graphical user interface of a healthcare provider computer to display the risk value, and based on the risk value, and transmitting one or more of an on-call staff alert or a welfare check request.

15. The non-transitory computer-readable data storage media of claim 14, the inference data comprising one or more of a Behavioral Health Score, Sleep Score, Social Score, Routine Score, and Physical Activity Score.

16. The non-transitory computer-readable data storage media of claim 14, the plurality of trained machine learning models each comprising an ensemble of decision trees that have been trained using training data to track a particular item on a patient health questionnaire for depression.

17. The non-transitory computer-readable data storage media of claim 16, the training data comprising a plurality of records each comprising attribute values for patient demographics and one or more inferences or behavioral summaries that have been derived from the raw sensor data, and one or more sets of responses to items of the patient health questionnaire.

18. The non-transitory computer-readable data storage media of claim 17, the training data comprising, for a particular patient that is associated with the particular mobile computing device, a plurality of sets of responses to the patient health questionnaire corresponding to a plurality of different days.

19. The non-transitory computer-readable data storage media of claim 17, the training data comprising, for a particular patient that is associated with the particular mobile computing device, a plurality of sets of responses to the PHQ-8 patient health questionnaire for depression and corresponding to a plurality of different days.

20. The non-transitory computer-readable data storage media of claim 16, further comprising sequences of instructions which when executed using one or more processors cause the one or more processors to execute, based on the inference data, selecting a digitally stored different questionnaire, transmitting the different questionnaire to the particular mobile computing device, receiving a plurality of responses corresponding to plural items in the different questionnaire, and updating the training data based on the responses.

21. The non-transitory computer-readable data storage media of claim 14, each model among the plurality of trained machine learning models comprising a classification model that is associated with a particular item of the patient health questionnaire, each classification model being configured to output a probability that, if given the patient health questionnaire, a response of a patient would exceed a particular threshold value for the particular item.

22. The non-transitory computer-readable data storage media of claim 21, the plurality of trained machine learning models comprising pairs of models, each of the pairs being associated with a common category of gender and activity, each of the models being trained on a particular item of the PHQ-8 patient health questionnaire for depression.

23. The non-transitory computer-readable data storage media of claim 22, the particular item comprising any of item “1”, “2”, “3”, or “4” of the PHQ-8 patient health questionnaire for depression.

24. The non-transitory computer-readable data storage media of claim 14, further comprising sequences of instructions which when executed using one or more processors cause the one or more processors to execute, based on the inference data, selecting and transmitting to the particular mobile computing device a plurality of suggestion messages each comprising a text suggestion for presentation on the particular mobile computing device.

25. The non-transitory computer-readable data storage media of claim 14, the plurality of raw sensor data comprising location data, pedometer data, activity data, and device data, the device data specifying any of device unlock events, device lock events, device lock screen display events, application launch events, application dismiss events.

26. The non-transitory computer-readable data storage media of claim 14, the activity data specifying physical interactions of a user with the particular mobile computing device including one or more gestures, taps, operation of hardware switches or controls.
